(19) United States
(12) Patent Application Publication
(10) Pub. No.: US 2023/0382819 A1
(43) Pub. Date: Nov. 30, 2023

(54) PROCESSES FOR SYNTHESIS OF ALPHA-EMITTING RADIOPHARMACEUTICALS

(71) Applicants: H. LEE MOFFITT CANCER CENTER AND RESEARCH INSTITUTE, INC., Tampa, FL (US); UNIVERSITY OF IOWA RESEARCH FOUNDATION, Iowa City, IA (US)

(72) Inventors: David L. MORSE, Valrico, FL (US); Thaddeus J. WADAS, Iowa City, IA (US); Darpan PANDYA, Coralville, IA (US); Narges TAFRESHI, Tampa, FL (US); Mikalai BUDZEVICH

(21) Appl. No.: 18/031,760

(22) PCT Filed: Oct. 14, 2021
(86) PCT No.: PCT/US2021/054897

§ 371 Date: Apr. 13, 2023

Related U.S. Application Data
(60) Provisional application No. 63/091,612, filed on Oct. 14, 2020.

(51) Int. Cl. C07B 59/00; A61K 51/08; A61K 45/06

(52) U.S. Cl. CPC C07B 59/008; A61K 51/083; A61K 45/06; C07B 2200/05; A61K 2121/00

(57) ABSTRACT

The present disclosure provides processes for the preparation of alpha emitting radiopharmaceutical compositions, in particular DOTATATE complexes of alpha emitting radionuclides, as well as use of such prepared compositions in the treatment of cancers.

### CROSS-REFERENCE TO RELATED APPLICATIONS

This application claims the benefit of priority to U.S. Provisional Application No. 63/091,612, filed Oct. 14, 2020, the disclosure of which is incorporated herein by reference in its entirety.

### TECHNICAL FIELD

This disclosure relates to processes for the synthesis of radiopharmaceuticals and use of said radiopharmaceuticals in the treatment of cancers, and more particularly to the preparation of DOTATATE complexes of alpha-emitting radionuclides and use of said complexes in the treatment of cancers.

### BACKGROUND

Neuroendocrine tumors (NETs) arise from the neuroendocrine system anywhere in the body including the lung, gastrointestinal tract, and pancreas (see Oronsky, B.; et al. Neoplasia 2017, 19 (12), 991-1002). A large fraction (25%) of well-differentiated NETs are of bronchopulmonary origin. Well- and moderately-differentiated lung NETs are referred to as lung carcinoids, and are divided into typical and atypical carcinoids as opposed to high-grade lung NETs that are divided into large cell neuroendocrine carcinoma and small cell lung cancer (SCLC) (see Hendifar, A. E.; et al. Arch Pathol Lab Med 2010, 134 (11), 1628-38).

While most NETs are indolent, the main causes of death are disseminated- or recurrent-disease. For the treatment of localized lung carcinoids, surgical resection is the standard of care. However, most NETs are inoperable or diagnosed at an advanced stage involving distant spread, thus requiring systemic therapies. Treatments for lung carcinoids are still limited and novel systemic therapies with greater efficacy and lower toxicity are greatly needed for treatment of patients with lung carcinoid tumors and metastases. There are a few systemic options (interferon, chemotherapy and molecularly targeted agents) including somatostatin analog (SSA) targeted β-emission therapies (TBTs) in patients with positive somatostatin receptor (SSTR) scintigraphy (see Brabander, T.; et al. Clin Cancer Res 2017, 23 (16), 4617-4624). SSA therapy is the first-line treatment option for NETs based on the results of the PROMID (see Rinke, A.; et al. J Clin Oncol 2009, 27 (28), 4656-63) and CLARINET (see Delavault, P.; et al. Journal of Clinical Oncology 2012, 30 (15_suppl), TPS4153-TPS4153) studies which demonstrated a significant prolongation of progression-free survival.

Overexpression of somatostatin receptors in patients with neuroendocrine neoplasms (NEN) is utilized for both diagnosis and treatment. Somatostatin receptor 2 (SSTR2) is widely expressed in pulmonary neuroendocrine tumors, including typical carcinoids, atypical carcinoids, large cell neuroendocrine, and SCLC (see O'Byrne, K. J.; et al. Eur J Cancer 1994, 30A (11), 1682-7; Papotti, M.; et al. Virchows Arch 2001, 439 (6), 787-97; and Taylor, J. E.; et al. Peptides 1994, 15 (7), 1229-36). Positive SSTR2 expression has been reported in approximately 66% of typical carcinoids, approximately 74% of atypical carcinoids, and 82% of metastatic lesions in patients with lung typical/atypical carcinoids, according to immunohistochemistry (IHC) of patient specimens (see Kanakis, G.; et al. Neuroendocrinology 2015, 101 (3), 211-22). SCLC tumor cells express SSTR2 (see Teijeiro, R.; et al. Cell Physiol Biochem 2002, 12 (1), 31-8), and 80%-100% can be visualized by octreotide scintigraphy. Therefore, SSAs with high affinity to SSTR2 can be used for targeting these types of tumors. Additionally, receptor internalization upon SSA binding and its subsequent recycling to the cell surface are beneficial for efficient tumor targeting (see Reubi, J. C.; et al. J Clin Endocrinol Metab 2010, 95 (5), 2343-50).

During the last decade, peptide receptor radionuclide therapy (PRRT) has been developed as a new treatment strategy for metastasized neuroendocrine tumors. PRRT is a form of molecular targeted therapy which is performed by using a target-specific peptide ligand that is coupled with a β-particle emitting radionuclide. The goal of PRRT is irradiation of tumor cells, via systemic delivery and direct binding to a specific target receptor that is overexpressed on the cell-surface membrane of primary tumors and metastases (see Lo Russo, G.; et al. Tumour Biol 2016, 37 (10), 12991-13003). Over many years of clinical use of PRRT with ¹⁷⁷Lu-DOTA chelate conjugated to SSAs has proved to be an effective therapy option for NETs, with tumor responses determined by radiological evaluation. DOTATATE is a compound made by conjugation of the metal chelator DOTA to the somatostatin-receptor (SSTR2) specific octreotate peptide. The systemic PRRT ¹⁷⁷Lu-DOTATATE (Lutathera; Advanced Accelerator Applications USA, Inc, Millburn, NJ) was recently approved by the US Food and Drug Administration (FDA) for the treatment of advanced SSTR2 positive gastroenteropancreatic NETs (GEP-NETs) (see Mittra, E. S., AJR Am J Roentgenol 2018, 211 (2), 278-285). The NETTER study group recently published results of the phase 3 neuroendocrine tumor therapy trial demonstrating the efficacy and safety of ¹⁷⁷Lu-DOTATATE in patients with advanced, progressive, somatostatin-receptor positive midgut neuroendocrine tumors and the treatment resulted in longer progression-free survival and a higher response rate relative to high-dose octreotide LAR (see Strosberg, J.; et al. N Engl J Med 2017, 376 (2), 125-135). An increased time to quality of life deterioration was also reported (see Strosberg, J.; et al. J Clin Oncol 2018, 36 (25), 2578-2584).

Although the efficacy of Lutathera in GEP-NETs is well documented, the limitation of ¹⁷⁷Lu-DOTATATE therapy is that 26-55% of patients only achieve stabilization of disease and a significant number (18-32%) are refractory to treatment. Moreover, those who achieve stabilization of disease invariably relapse 2 to 3 years after starting treatment (see Ballal, S.; et al. Clin Nucl Med 2017, 42 (11), e457-e466; Bodei, L.; et al. Eur J Nucl Med Mol Imaging 2011, 38 (12), 2125-35; and Navalkissoor, S.; et al. Neuroendocrinology 2019, 108 (3), 256-264). ¹⁷⁷Lu is a β-particle emitting radionuclide and there is evidence that systemically administered targeted α-particle emitting radiotherapies have greater efficacy and reduced toxicity relative to targeted β-particle emitting therapies (see Andersson, H.; et al. Anticancer Res 2001, 21 (1A), 409-12; Miederer, M.; et al. Clin Cancer Res 2008, 14 (11), 3555-61; Milenic, D.; et al. Cancer Biother Radiopharm 2004, 19 (2), 135-47; Song, H.; et al. Cancer Res 2009, 69 (23), 8941-8; Wild, D.; et al. Cancer Res 2011, 71 (3), 1009-18; and Tafreshi, N. K.; et al. Molecules 2019, 24 (23)).

Many strategies are being explored to try to improve on and optimize the effectiveness of PRRT. Some of these strategies include intra-arterial treatments (to better target liver metastases) (see Kratochwil, C.; et al. Endocr Relat Cancer 2011, 18 (5), 595-602), using dosimetry to ensure adequate tumor dosing (see Bison, S. M.; et al. Clin Transl Imaging 2014, 2, 55-66), combining ¹⁷⁷Lu-DOTATATE and ⁹⁰Y PRRT to target both small and large tumors (see Villard, L.; et al. J Clin Oncol 2012, 30 (10), 1100-6), using chemotherapy as a PRRT radiosensitizer (see Claringbold, P. G.; et al. Cancer Biother Radiopharm 2012, 27 (9), 561-9), a somatostatin receptor (SSR) antagonist PRRT (which has a higher SSR binding affinity) (see Wild, D.; et al. J Nucl Med 2014, 55 (8), 1248-52), and targeted α-particle therapy (TAT) (see Ballal, S.; et al. Eur J Nucl Med Mol Imaging 2020, 47 (4), 934-946; and Kratochwil, C.; et al. Eur J Nucl Med Mol Imaging 2014, 41 (11), 2106-19).

The therapeutic application of α-particle emitting isotopes coupled to tumor-homing peptides or antibodies is an innovative approach in cancer therapy. Alpha (α)-emissions consist of di-cationic helium nuclei (He²⁺) and are characterized by a high linear energy transfer rate leading to an extremely high cytotoxic activity on the cellular level, and a short range in tissue, reducing side effects in normal tissues.

Actinium-225 (²²⁵Ac) is an α-particle emitting isotope that has demonstrated significant utility in systemic targeted radionuclide therapy and decays with a 10-day half-life via a complex decay scheme, emitting four a particles and depositing a high energy levels (28 MeV) locally in tissue (see Ma, D.; et al. Appl Radiat Isot 2001, 55 (5), 667-78). Hence, ²²⁵Ac has been described as an in vivo α-particle generator (see Borchardt, P. E.; et al. Cancer Res 2003, 63 (16), 5084-90). Compared to β particles, a particles have a 160 to 600 fold greater linear energy transfer and a shorter range in solid tissue, <100 μm for a particles relative to a few mm for β-particles, making it ideal for treatment of metastases or killing small clusters of tumor cells with minimal damage to surrounding normal tissues. Unlike β emissions, α emissions do not necessarily rely on generation of free-radicals to generate DNA damage. The energy deposited is great enough to directly cause DNA double-strand breaks alone, albeit significant levels of free radicals are generated in the emission tracks of oxygenated tissue (see Nonnekens, J.; et al. Cancer Biother Radiopharm 2017, 32 (2), 67-73). This potentially enables TATs to evade a common mechanism of radiation resistance (free radical scavenging). Moreover, it can be easily chelated by DOTA, which allows for attachment to a tumor targeting moiety (see Pandya, D. N.; et al. Theranostics 2016, 6 (5), 698-709). In addition, ²²⁵Ac-DOTA-chelates have shown sufficient stability to serve as a radionuclide delivery system for ²²⁵Ac-targeted therapy (see Tafreshi, N. K.; et al. J Nucl Med 2019, 60 (8), 1124-1133).

There is a clear need for processes for the preparation of alpha emitting radiopharmaceutical compositions which may then be used in the treatment of cancers. This present disclosure answers this as well as other needs.

### SUMMARY

Disclosed herein are processes for the preparation of alpha emitting radiopharmaceutical compositions, as well as methods of treating cancers using the compositions prepared by these methods. In particular, processes are provided for the preparation of DOTATATE chelates of alpha emitting radionuclides (for example, ²²⁵Ac) by allowing for a period in which the composition can reach secular equilibrium prior to administration to a subject for treatment. Also provided are kits that contain the necessary components in which to perform the processes described herein.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DESCRIPTION OF DRAWINGS

Many aspects of the present disclosure can be better understood with reference to the following drawings. The components in the drawings are not necessarily to scale, emphasis instead being placed upon clearly illustrating principles of the present disclosure. Moreover, in the drawings, like reference numerals designate corresponding parts throughout the several views.

FIG. 1 depicts a competitive binding assay of La³⁺-DOTATATE. The binding affinity of La³⁺-DOTATATE was calculated to be 19.00±9.2 nM Ki (n=3 repeats). In this whole-cell assay, HEK293/SSTR2 cells were used and 50 nM of Eu³⁺-DOTATATE was used as the competing ligand.

FIGS. 2A-2B depict SSTR2 expression on H69 and H727 cells. (FIG. 2A) qRT-PCR. Note the Log10 scale for the expression level. (FIG. 2B) ICC using DAPI, WGA and anti-SSTR2 antibody.

FIGS. 3A-3F depict toxicity of ²²⁵Ac-DOTATATE. (FIG. 3A) Percent weight gain, (FIG. 3B) BUN (reference range: 18-29 mg/dl), (FIG. 3C) blood creatinine (reference range: 0.2-0.8 mg/dL), (FIG. 3D) ALT (reference range: 28-132 U/L), (FIG. 3E) AST (reference range: 59-247 U/L) and (FIG. 3F) ALKP (reference range: 62-209 U/L) following i.v. injection of a range of activities and saline into cohorts of BALB/c mice.

FIGS. 4A-4B depict histological appearance of kidney from a mouse administered (FIG. 4A) saline or (FIG. 4B) 179.0 kBq. (FIG. 4A) Kidney histology of a saline administered mouse, without significant abnormalities, where glomeruli are normocellular, with open capillary loops, and tubular epithelium is uniformly cuboidal, with round nuclei, and a prominent brush border. (FIG. 4B) Kidney histology of a 179.0 kBq administered mouse, with chronic progressive nephropathy. Extensive tubular cell regeneration, diffuse fibrosis, mild mononuclear inflammatory cell infiltrates, and thickened, hypercellular glomerular tufts are evident.

FIGS. 5A-5C depict biodistribution of (FIG. 5A)²²⁵Ac, (FIG. 5B)²²¹Fr, and (FIG. 5C)²¹³Bi following administration of ²²⁵Ac-DOTATATE in BALB/c mice. Animals were intravenously administered 74 kBq (±5%) of ²²⁵Ac a activity in the syringe.

FIG. 6 depicts radiation dosimetry of ²²⁵Ac and daughters following administration of ²²⁵Ac-DOTATATE in BALB/c mice.

FIG. 7A-7J depict efficacy of ²²⁵Ac-DOTATATE in SCID mice bearing H69 and H727 tumors. The treated animals were intravenously injected with a single bolus of 148.7 kBq and 144.3 kBq mean ²²⁵Ac activity for H69 and H727 cohorts, respectively, or saline solution at 8 days after tumor cells inoculation. Representative images of mice bearing (FIG. 7A) H69 and (FIG. 7B) H727 tumors (outlined), 25 days post inoculation. Tumor growth volumes of (FIG. 7C) H69 and (FIG. 7D) H727 tumors relative to saline controls (arrow indicates the day of injection). Kaplan-Meier plots of (FIG. 7E) % H69 and (FIG. 7F) H727 tumor bearing mice that have reached the experimental endpoint. Representative SSTR2 IHC images of treated and control (FIG. 7G) H69 tumors and (FIG. 7H) H727 tumors at experimental endpoint. Quantified SSTR2 IHC expression in treated and control (FIG. 7I) H69 tumors and (FIG. 7J) H727 tumors.

FIG. 8 depicts the decay chain of Actinium-225 (²²⁵Ac).

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teaching presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of the disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or types of aspects described in the specification.

All publications mentioned herein are incorporated by reference to disclose and describe the methods and/or materials in connection with which the publications are cited. The publications discussed herein are provided solely for their disclosure prior to the filing date of the present application. The dates of publication provided herein can be different from the actual publication dates, which can require independent confirmation.

It is also to be understood that the terminology used herein is for the purpose of describing particular aspects only and is not intended to be limited. Unless defined otherwise, all technical and scientific terms used herein have the same meaning as commonly understood by one of ordinary skill in the art to which the disclosed compositions and methods belong. It would be further understood that terms, such as those defined in commonly used dictionaries, should be interpreted as having a meaning that is consistent with their meaning in the context of the specification and relevant art and should not be interpreted in an idealized or overly formal sense unless expressly defined herein. Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as refereed to, but does not preclude the presence or addition of one or more features, integers, steps, or components, or groups thereof. Moreover, each of the terms “by”, “comprising”, “comprises”, “comprised of”, “including”, “includes”, “included”, “involving”, “involves”, “involved”, and “such as” are used in their open, non-limiting sense and may be used interchangeably. Further, the term “comprising” is intended to include examples and aspects encompassed by the terms “consisting essentially of” and “consisting of”. Similarly, the term “consisting essentially of” is intended to include examples encompassed by the term “consisting of”.

As used in the specification and the appended claims, the singular forms “a”, “an” and “the” include plural referents unless the context clearly dictates otherwise. Thus, for example, reference to “a composition”, “a radionuclide”, or “a process”, includes but is not limited to, two or more compositions, radionuclides, or processes, and the like.

It should be noted that ratios, concentrations, amounts, and other numerical data can be expressed herein in a range format. It will be further understood that the endpoints of each of the ranges are significant both in relation to the other endpoint, and independent of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further aspect. For example, if the value “about 10” is disclosed, then “10” is also disclosed.

When a range is expressed, a further aspect includes from the one particular value and/or to the other particular value. For example, when the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about x to y”, where x and y are numerical values, includes “about x to about y”.

It is to be understood that such a range format is used for convenience and brevity, and thus, should be interpreted in a flexible manner to include not only the numerical values explicitly recited as the limits of the range, but also to include all the individual numerical values or subranges encompassed within that range as if each numerical value and subrange is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the subranges (e.g., about 0.5% to about 1.1%, about 5% to about 2.4%, about 0.5% to about 3.2%, and about 0.5% to about 4.4%, and other possible subranges) within the indicated range.

As used herein, the terms “about”, “approximate”, “at or about”, and “substantially” mean that the amount or value in question can be the exact value or a value that provides equivalent results or effects as recited in the claims and taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error, and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about”, “approximate”, or “at or about” whether or not expressly stated to be such. It is understood that where “about”, “approximate”, or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, the term “composition” is intended to encompass a product comprising the specified ingredients in the specified amounts, as well as any product which results, directly or indirectly, from combination of the specified ingredients in the specified amounts.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Processes for the Preparation of Alpha Emitting Radiopharmaceutical Compositions

The present disclosure provides processes for the preparation of alpha emitting radiopharmaceutical compositions. The disclosed processes offer several advantages. They reduce the number of preparative steps and time necessary to synthesize ²²⁵Ac- or other alpha-particle emitter-based radiotherapies, which should translate into reduced production costs. Additionally, the disclosed methods facilitate the development of standardized kit technology that may allow greater access to TAT at clinical sites where radiochemistry resources or technical expertise are unavailable. Finally and most importantly, the disclosed revised protocol allows radiopharmaceuticals to be generated with significantly improved specific activity, which is an important outcome criterion in radiochemistry (see Lapi, S. E.; et al. Nucl Med Biol 2012, 39 (5), 601-8; Wessels, B. W.; et al. Journal of Nuclear Medicine 1983, 24 (5), P95; Wessels, B. W.; et al. Med Phys 1984, 11 (5), 638-45; Mausner, L. F.; Srivastava, S. C., Med Phys 1993, 20 (2 Pt 2), 503-9; Bonardi, M. L.; de Goeij, J. J., J Radioanal Nucl Chem 2005, 263 (1), 87-92; and de Goeij, J. J.; Bonardi, M. L., J Radioanal Nucl Chem 2005, 263 (1), 13-18). Injection of a radiopharmaceutical that exhibits a high amount of radio-activity per unit mass reduces the possibility of a host response after agent injection and ensures favorable accumulation at the tumor site. Furthermore, radiopharmaceuticals with high and non-variable specific activity and formulated with clinically approved antioxidants should face less scrutiny by regulatory agencies responsible for ensuring patient safety and agent efficacy in the clinical setting.

Thus in one aspect, a process is provided for manufacturing an alpha emitting radiopharmaceutical solution comprising:
combining DOTATATE with an alpha emitting radionuclide and at least one stabilizer against radiolytic degradation in an aqueous medium to form a solution;
heating the solution to a temperature of greater than about 50 degrees Celsius for a period of at least 30 minutes to form a chelate solution;
cooling the chelate solution to about 25 degrees Celsius and maintaining at said temperature until secular equilibrium is reach for the alpha emitting radionuclide.

“DOTATATE” as described herein, also referred to as DOTA-TATE, DOTA-octreotate, oxodotreotide, DOTA-(Tyr³)-octreotate, or DOTA-O-Tyr³-octreotate), is an eight amino acid peptide SSR antagonist covalently bound to a DOTA bifunctional chelator and has the chemical structure:

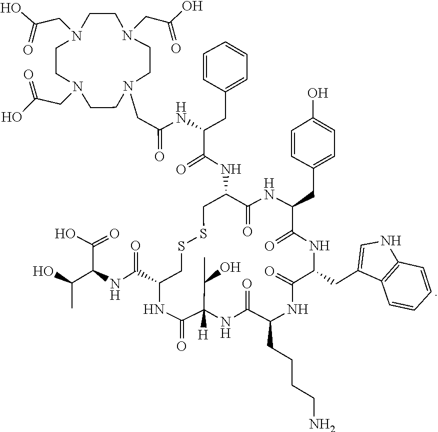 [SMILES: C[C@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O]

The term “alpha-particle emitting radionuclide” as used herein encompasses nuclei with a single (alpha) mode of radioactive decay and also nuclei with multiple decay modes where at least a portion of the nuclei of this isotope decay by alpha emission. Where a nucleus has more than one emission mode, it is preferable that at least 1% will decay by alpha emission, preferably at least 10%, or more preferably at least 30% will decay by alpha-emission. In one embodiment, substantially all nuclei will decay by alpha emission. Where a “branching” alpha emitter having two or more decay modes produces a relatively low proportion of alpha particles, this may however also be an “indirect” alpha emitter since the product of non-alpha decay may result (directly or indirectly) in a further alpha emitter.

The term “alpha particle emitting radionuclide” as used herein is also used to indicate, where context permits, an “indirect” alpha-emitting radionuclide. Such indirect alpha emitters may be radioisotopes which do not themselves undergo alpha decay to a significant extent but decay by another mode (e.g., by beta-emission) to form an alpha emitter. Preferably, this alpha emitter will be the direct daughter product of the “indirect” alpha emitter but may be the result of more than one non-alpha decay. Preferably the alpha-emitter formed from the decay of an “indirect” alpha emitter will have a short half-life (e.g., less than 24 hours, more typically less than 1 hour and preferably less than 10 minutes). Examples of indirect alpha emitters include ²¹²Pb, ²¹²Bi and ²¹³Bi (the latter two also being “branching” alpha emitters in themselves).

The half-life of suitable alpha emitters will generally be sufficient to allow their preparation, transport and limited storage prior to use as a radiotherapeutic but will typically not be so long as to pose a long-term health risk to the subject administered such therapeutics if a certain amount of the isotope is retained in the body during and after treatment. Thus, suitable alpha emitters (including indirect alpha emitters) will typically have half-lives of at least 30 minutes, preferably at least 6 hours, and more preferably at least 1 day. Most preferred alpha emitters would have a half life of at least 3 days.

In order to avoid long term exposure, the half-life of the alpha emitters should generally be less than a year, preferably less than 6 months and more preferably less than 1 month. The body will also potentially be exposed to radiation from any alpha-emitting daughter generated by subsequent decay. Thus, where the decay chain from the administered alpha emitter includes one or more alpha-emitters, it is preferable that no isotope generated in that decay chain, up to and including the last alpha-emitting isotope before the formation of a stable nucleus, has a half life of greater than 1 year. Most preferably this should be no greater than 6 months and most preferably no greater than 1 month.

Representative examples of alpha emitting radionuclides as may be used in the present disclosure include, but are not limited to, ²¹¹At, ²¹²Bi, ²¹²Pb, ²¹³Bi, ²²³Ra, ²²⁴Ra, ²²⁵Ra, ²²⁵Ac, and ²²⁷Th. In particular embodiments, the alpha emitting radionuclide comprises ²²⁵Ac. In some embodiments, the alpha emitting radionuclide may be administered in the form of a salt, for example a chloride salt or a nitrate salt.

In some embodiments, the radionuclide is present in a final concentration in the chelate solution that provides a volumetric radioactivity of at least 100 MBq/mL, for example at least 250 MBq/mL. In some embodiments, the radionuclide is present in a final concentration in the chelate solution that provides a volumetric radioactivity from 100 to 1000 MBq/mL, for example from 100 to 750 MBq/mL, from 100 to 500 MBq/mL, from 100 to 250 MBq/mL, from 250 to 1000 MBq/mL, from 250 to 750 MBq/mL, from 250 to 500 MBq/mL, from 500 to 1000 MBq/mL, from 500 to 750 MBq/mL, or from 750 to 1000 MBq/mL. In preferred embodiments, the radionuclide is present in a final concentration in the chelate solution that provides a volumetric radioactivity from 250 to 500 MBq/mL.

The stabilizer against radiolytic degradation is included in the presently disclosed solutions to reduce the cleavage of the chemical bonds of the molecules which form the radiopharmaceutical product due to the high energy emissions released during the constant decay of the included radionuclide. Stabilizer which may be used in accordance with the present disclosure may be selected from gentisic acid (2,5-dihydroxybenzoic acid) or salts thereof, ascorbic acid (L-ascorbic acid, vitamin C) or salts thereof (e.g., sodium ascorbate, methionine, N-acetyl cysteine, histidine, melatonin, ethanol, and Se-methionine. In preferred embodiments, the stabilizer is selected from gentisic acid or salts thereof and ascorbic acid or salts thereof. In some embodiments, the stabilizer is present in a final concentration in the chelate solution of at least 0.2 mg/mL, at least 0.5 mg/mL, at least 1.0 mg/mL, or at least 2.7 mg/mL.

“Secular equilibrium” is defined as the point in which the quantity of a radioactive isotope remains constant because its product rate equals its decay rate. In the radiopharmaceutical solutions described herein, secular equilibrium is considered to be reached when the quantity of daughter isotopes for the alpha emitting radionuclide are considered to remain nearly constant. In some embodiments, the chelate solution is maintained at about 25 degrees Celsius for at least 4 hours, at least 8 hours, at least 12 hours, at least 18 hours, or at least 24 hours until secular equilibrium is reached.

In some embodiments, the process further comprises filtering the chelate solution to remove any residual solid components prior to administration to a subject.

Owing to the large amount of energy deposited by alpha particles, it is important to quantify the radioactivity of the targeted radiopharmaceutical initially injected and its subsequent biodistribution, pharmacokinetics, and radiation dose deposited in various tissues. As a result of its short path length, the direct measurement of alpha particles' activity in tissues is not possible in most, if not all, preclinical scenarios. If a parent metastable radioisotope also emits gamma rays, indirect methods of detection such as gamma spectroscopy can be used to estimate activity using scintillation detectors and well-type ion chambers that are commonly available in research laboratories and nuclear medicine clinics.

In particular, ²²⁵Ac has a 10-day half life and decays via six daughter radionuclides resulting in a net of four alpha particles per Actinium disintegration (see FIG. 8). ²²⁵Ac and two of its daughters, ²²¹Fr and ²¹³Bi, also decay with accompanying isomeric gamma photons. Using gamma detection systems, such as ion chambers or scintillation detectors, it is possible to indirectly determine the alpha activity by gamma ray abundance per decay conversions.

In the clinic, ion chambers are readily available and their use is the standard of practice for checking activity for diagnostic and therapeutic agents. An ion chamber reading does not discriminate the collected charge between the parent and daughter radionuclides because it does not provide energy identification. In these situations gamma spectroscopy using scintillation or semiconductor detectors, such as a sodium iodide doped with thallium scintillation detector [NaI(Tl)] or a high purity germanium (HPGe) detector, can be used. With this approach, it is possible to determine the activity of each gamma emitting daughter, which gives more information on how the decay species behave.

While HPGe seems to be the attractive option owing to its superior energy resolution, its performance suffers as a result of its low detection efficiency, cost, and requirement of sophisticated cooling systems. Despite the advantages of the NaI(Tl), dead time losses at higher activities and poor energy resolution may also provide incorrect activity measurements, leading to underestimation or overestimation of radiation dose.

In pre-clinical and clinical biodistribution studies, activity measurements of collected blood and tissues are used to calculate pharmacokinetics and radiation dosimetry. Therefore, accurate measurements for ²²⁵Ac and daughters are needed. Hence, scintillation detector measurements are needed to generate these spectra. Alpha radiation dosimetry is performed using the methods recommended by the Committee on Medical Internal Radiation Dose. It is essential that these measurements are accurate because the dosimetry estimates are then extrapolated to human estimations and, therefore, serve as the fundamental basis for all patient safety measures.

Scintillation detectors are calibrated by analyzing three aspects of the detector: energy, resolution, and efficiency. Once the detector is fully calibrated, it can be used to perform gamma spectroscopic measurements in order to determine the activity of radioactive samples. Ion chambers are calibrated by measuring a sample of the radiopharmaceutical in question with known activity, correcting for decay, and applying a calibration factor. The American National Standards Institute (ANSI) recommends that the applied calibration factor adjusts the measurement to within ±10% of the known activity. Although reference standards have been developed for isotopes used in internal radiotherapy, ²²⁵Ac standards are currently under development by NIST and are not yet available.

While there are evident limitations in making clinical predictions when translating a radiopharmaceutical from mice to humans, it is important to minimize these limitations that are the result of instrumentation. As described in Tichacek et al. Molecules 2019, 24:3397 and incorporated herein in its entirety for all purposes, a threshold is identified above which the scintillation detector cannot accurately measure the activities of ²²⁵Ac and daughters. An activity response relationship between the ion chamber and scintillation detector measurements is reported and used in a method to improve activity determination above the threshold by correcting scintillation detector measurements via Monte Carlo simulations. These corrections improve the dosimetry of pre-clinical work and ultimately facilitate the translation of new radiopharmaceuticals to the human clinic.

In another aspect, a radiopharmaceutical composition is provided formed by the processes described herein.

### Methods of Treatment

Further provided herein are methods of treating or preventing cancer in a subject, comprising administering to the subject an effective amount of a radiopharmaceutical composition prepared according to the processes described herein. Methods of killing a tumor cell are also provided herein, said methods comprising contacting a tumor cell with an effective amount of a radiopharmaceutical composition prepared according to the processes described herein. Also provided herein are methods of radiotherapy of tumors, comprising administering to a subject a radiopharmaceutical composition prepared according to the processes described herein, which delivers ionizing radiation to the tumor.

Also disclosed are methods for treating oncological disorders in a patient. In one embodiment, an effective amount of a radiopharmaceutical composition described herein is administered to a patient having an oncological disorder and who is in need of treatment thereof. The disclosed methods can optionally include identifying a patient who is or can be in need of treatment of an oncological disorder. The patient can be a human or other mammal, such as a primate (e.g., monkey, chimpanzee, ape, etc., dog, cat, cow, pig, or horse, or other animals having an oncological disorder.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstram macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

The present disclosure also provides methods for the treatment of neuroendocrine tumors. Neuroendocrine tumors (NETs) are tumors that arise from cells of the endocrine (hormonal) and nervous systems. NETs include a group of tumors with a wide range of morphologic, functional, and behavioral characteristics. These tumors are generally slow growing but have the potential to spread, primarily to the liver, and when they do, they can be life threatening and difficult to treat with current modalities.

Neuroendocrine tumors have traditionally been classified by the site of their origin. In certain embodiments, the NET is selected from group consisting of pancreatic neuroendocrine tumors (pNETS) and carcinoid tumors of the lung, stomach, duodenum, jejunum, ileum, colon and rectum. In further embodiments, the NET is selected from the group consisting of neuroendocrine tumors of the ovary, thymus, thyroid medulla, adrenal glands (e.g., pheocromocytoma) and paraganglia (paraganglioma). In certain embodiments, the NET treated by the methods described herein is small cell lung cancer (SCLC). In other embodiments, the NET is a non-small cell lung cancer (NSCLC). In some embodiments, the NET is a pancreatic neuroendocrine tumor (PET) or carcinoid tumor. In certain embodiments, the NET is non-small cell lung cancer, a pancreatic cancer, or a thyroid cancer.

Neuroendocrine tumors are also classified by grade and differentiation (see Phan et al., Pancreas, 2012, 39(6):784-798). In certain embodiments, the neuroendocrine tumor is a well differentiated, low grade tumor. In certain embodiments, the neuroendocrine tumor is a moderately differentiated, intermediate grade tumor. In certain embodiments, the neuroendrocrine tumor is a poorly differentiated, high grade tumor. In one embodiment, low grade tumors are characterized by <2 mitoses per 10 HPF (high power fields) and no necrosis. In one embodiment, intermediate grade tumors are characterized by 2-10 mitoses per 10 HPF (high power fields) or foci of necrosis. In one embodiment, high grade tumors are characterized by >10 mitoses per 10 HPF (high power fields).

In other embodiments, neuroendocrine tumors can be divided based on the WHO classification 2000 or 2010 into Neuroendocrine Tumors Grade 1-Grade 2(or well-differentiated endocrine tumor or carcinoma (WDET/WDEC)), Neuroendocrine Carcinoma Grade 2 or Poorly Differentiated Endocrine Carcinoma/Small-Cell Carcinoma (PDEC), Mixed Adenoneuroendocrine Carcinoma (MANEC) and Hyperplastic and Preneoplastic Lesions. According to ENETSIWHO/AJCC classification systems Tumors G1 are those with Ki67 index≤2% or MI (mitotic count)<2, Tumors G2 are those with Ki67 index within 3-20% and MI=2-20, and Tumors G3 are those with Ki67 index≥20% and MI>20.

Neuroendocrine tumors are also classified as functional and non-functional NETs. NETs are considered functional when a specific clinical syndrome is induced due to excessive production of hormones by the tumor cells. Examples of functional NETs include, but are not limited to, carcinoid tumors, which can result in carcinoid syndrome, and functional pNETs, for example, insulinomas, gastrinomas, vasoactive intestinal peptide (VIP)omas, glucagonomas, and somatostatinomas.

Non-functional NETs are not associated with a clinical syndrome due to excessive production of hormones by the tumor cells, but can still produce symptoms related to the presence of the tumor or its metastasis (e.g., abdominal pain or bloating). In certain embodiments, the neuroendocrine tumor is a functional NET. In certain embodiments, the neuroendocrine tumor is a non-functional NET. In certain embodiments, the neuroendocrine tumor is selected from the group consisting of functional carcinoid tumor, insulinoma, gastrinoma, vasoactive intestinal peptide (VIP)oma, glucagona, serotoninoma, histaminoma, ACTHoma, pheocromocytoma, and somatostatinoma. In certain embodiments, the neuroendocrine tumor is NSCLC.

In certain embodiments, the neuroendocrine tumor is a primary tumor. In alternative embodiments, the neuroendocrine tumor is a metastatic tumor. In certain embodiments, the neuroendocrine tumor has not spread outside the wall of the primary organ. In certain embodiments, the neuroendocrine tumor has spread through the wall of the primary organ and to nearby tissues, such as fat, muscle, or lymph nodes. In certain embodiments, the neuroendocrine tumor has spread to tissues or organs far away from the primary organ, for example to the liver, bones, or lungs.

In certain embodiments, the neuroendocrine tumor is refractory to treatment. In some embodiments, the cancer or tumor may be chemorefractory (i.e., resistant to one or more forms of chemotherapy). In some embodiments, the cancer or tumor is resistant to treatment with a somatostatin analog. In some embodiments, the cancer or tumor is resistant to treatment with a kinase inhibitor. In some embodiments, the cancer or tumor is resistant to treatment with an inhibitor of the PD-1/PD-L1/CTLA-4 pathway.

In some embodiments, the radiopharmaceutical compositions formed by the processes described herein may be administered in combination with one or more additional therapeutic agents. In some embodiments, the one or more additional therapeutic agents may comprise an agent used to treat cancer, i.e., a cancer drug or anti-cancer agent. Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, rnedroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof. According to some aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

The compositions prepared by the processes described herein may be typically administered to a subject in need thereof by an intravascular route, preferably by intravenous injection or by intratumor injection, preferably in the form of a sterile nonpyrogenic solution.

In some embodiments, the radiopharmaceutical compositions prepared by the processes described herein is delivered to a subject at a therapeutically effective amount comprised between 2000 and 10000 MBq, for example from 2000 to 9000 MBq, from 2000 to 8000 MBq, from 2000 to 7000 MBq, from 2000 to 6000 MBq, from 2000 to 5000 MBq, from 2000 to 4000 MBq, from 2000 to 3000 MBq, from 3000 to 10000 MBq, from 3000 to 9000 MBq, from 3000 to 8000 MBq, from 3000 to 7000 MBq, from 3000 to 6000 MBq, from 3000 to 5000 MBq, from 3000 to 4000 MBq, from 4000 to 10000 MBq, from 4000 to 9000 MBq, from 4000 to 8000 MBq, from 4000 to 7000 MBq, from 4000 to 6000 MBq, from 4000 to 5000 MBq, from 5000 to 10000 MBq, from 5000 to 9000 MBq, from 5000 to 8000 MBq, from 5000 to 7000 MBq, from 5000 to 6000 MBq, from 6000 to 10000 MBq, from 6000 to 9000 MBq, from 6000 to 8000 MBq, from 6000 to 7000 MBq, from 7000 to 10000 MBq, from 7000 to 9000 MBq, from 7000 to 8000 MBq, from 8000 to 10000 MBq, from 8000 to 9000 MBq, or from 9000 to 10000 MBq.

### Kits

Kits for practicing the processes and methods described herein are further provided. by “kit” is intended any manufacture (e.g., a package or container) comprising at least one reagent, e.g., one component of the radiopharmaceutical composition as described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a composition comprising a compound as used in the processes or methods described herein in one or more containers. The disclosed kits can optionally include pharmaceutically acceptable carriers and/or diluents. In one embodiment, a kit includes one or other components, adjuncts, or adjuvants as described herein. In another embodiments, a kit includes instructions or packaging materials that describe how to administer a composition of the kit. Containers of the kit can be any suitable material, e.g., glass, plastic, metal, etc., and of any suitable size, shape, or configuration. In one embodiment, one or more of the components used in the processes described herein is provided as a solid, such as a powder form. In another embodiment, one or more of the components used in the processes described herein is provided in the kit as a liquid or solution. In one embodiment, the kit comprises an ampoule or syringe containing one or more of the components described herein in liquid or solution form.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1. Pre-Clinical Evaluation of ²²⁵Ac-DOTATATE for Treatment of Lung Tumors

In this Example, ²²⁵Ac-DOTATATE was generated which is a targeted α-particle emitting derivative of ¹⁷⁷Lu-DOTATATE for treatment of lung carcinoid tumors. Its biodistribution (BD), radiation dosimetry (RD), and toxicity in normal BALB/c mice was then determined. To evaluate its efficacy, ²²⁵Ac-DOTATATE was tested in SCID mice bearing somatostatin receptor 2 (SSTR2) positive human lung carcinoid and SCLC xenograft tumors.

### Materials and Methods

Cell culture. NCI-H69 human SCLC tumor cells and NCI-H727 human lung carcinoid tumor cells were purchased from American Type Culture Collection, expanded for 2 passages and cryopreserved. HEK293 cells were grown in DMEM/F12 medium (Life Technology) and the NCI-H69 and NCI-H7272 cells were grown in RPMI-1640 Medium, (Life Technologies), 10% FBS (Life Technologies), 100 units/mL penicillin, 100 mg/mL streptomycin in 5% CO₂ at 37° C. Cells were authenticated using short tandem repeat (STR) DNA typing according to ATCC's guidelines (see Reid, Y. et al., Authentication of Human Cell Lines by STR DNA Profiling Analysis. In Assay Guidance Manual, Bethesda (MD), 2004) monitored by microscopy to assure maintenance of their original morphology, tested for mycoplasma contamination using the MycoAlert kit (Lonza) and experiments used cells of passage numbers <25.
Generation of stably transfected HEK293 cells bearing the SSTR2 gene. The HEK293 cells were seeded in 6 well plate at a density of 250,000 cells/well and incubated overnight. The cells were then transfected with 2 μg of SSTR2 (NM_001050) Human Tagged ORF Clone in pCMV6 vector with Neomycin selection marker (Origene) using the FuGENE HD Transfection Reagent (Promega). Two days after transfection, the cells were treated with 600 μg/mL of G418 (Life Technology). After 2 weeks, resistant colonies appeared. Large colonies were selected and transferred to individual plates. To determine the clone with the highest expression of SSTR2, quantitative real-time RT-PCR (qRT-PCR) was done using the same primers described below. The clone with the highest SSTR2 expression was selected and maintained in medium containing 400 μg/mL of G418.
qRT-PCR and Immunocytochemistry (ICC). To verify the expression of hSSTR2, qRT-PCR and ICC were performed. RNA of the H69 and H727 cells were extracted using RNeasy kit (Qiagen). Two sets of human SSTR2 primers were designed using Gene Runner Software for Windows version 3.05: Set 1 primers; Forward, 5′-AACCAGACAGAGCCGTACTA-3′ and reverse, 5′-GCATAGCGGAGGATGACA-3′ and Set 2 primers: Forward, 5′-GCTGGCTTCCCTTCTACATATT-3′ and Reverse, 5′-GAGGACCACCACAAAGTCAA-3′. B-actin was used for normalization as described previously (see Morse, D. L., et al. Anal Biochem 2005, 342 (1), 69-77).

For ICC, since H69 cells are grown in suspension, the coverslip was coated with 0.25 mg/ml poly-D-lysine (Sigma). H69 and H727 cells were plated at a cell density of 1×10⁴ cells/well on glass coverslips placed at the bottom of culture wells and incubated for 16 h. Cells were then treated with 30 μg/mL anti-SSTR2 antibody (Sigma, HPA007264) and 5.0 μg/mL of WGA (Invitrogen) at 4° C. for 10 min, washed 3 times with PBS, fixed with cold methanol/acetone (1:1), and air-dried for 20 min. Plates were washed 3 times with warm PBS and incubated with 1:2000 secondary antibody (Alexa-Fluor 555 goat anti rabbit IgG, Invitrogen). After three washes with PBS, coverslips were mounted with mounting medium and DAPI. Samples were viewed using an automated Zeiss Observer Z.1 inverted microscope using 40×/1.3 NA oil immersion objectives through narrow bandpass DAPI and FITC/A488 Chroma filter cubes and Nomarski Differential Interference Contrast polarizing and analyzing prisms. Images were produced using the AxioCam MRm CCD camera and Axiovision v 4.6 software suite (Carl Zeiss Inc., Germany). Image Pro Plus 6.2 software (Mediacybernetics Inc.) was used to measure the probe-related fluorescence intensity per image.

Loading of DOTATATE with Lanthanum and Europium. DOTA-(Tyr3)-octreotate (DOTATATE) with ≥98% purity was purchased from Bachem. To load the DOTATATE with lanthanum (La³⁺), LaCl₃·7H₂O (3.9 mg, 10.5 μmol) was added to a solution of DOTATATE (5.0 mg, 3.5 μmol) in 1 mL of 0.1 M AcONa buffer (pH 6.0). The mixture was stirred at room temperature for 12 h. After completion of the reaction checked by HPLC, the mixture went through the C18 column to yield the final product (˜3.3 mg). For Eu³⁺ loading, EuCl₃·6H₂O (1.6 mg, 4.2 μmol) was added to a solution of DOTATATE (2 mg, 1.4 μmol) in 5 mL H₂O and 0.1 mL DMSO. The mixture was stirred at room temperature for 12 h. After completion of the reaction checked by HPLC, the mixture went through the C18 column to yield the final product (1.9 mg). For both chelates, mass was determined by HRMS (ESI) and purity determined by HPLC.

Binding Assay. HEK293 cells engineered to express SSTR2 and the receptor number was determined by using time-resolved fluorescence (TRF) saturation binding assay as described before (see Xu, L.; et al. Bioorg Med Chem Lett 2010, 20 (8), 2489-92), except that Eu³⁺-DOTATATE was used as the test ligand and 5 μM of La³⁺-DOTATATE was used as the blocking ligand. Also, a modification to the protocol was applied due to the use of the stronger chelator, DOTA, as follows: After the ligand incubation step and wash step, the cells were incubated with 50 μl of 2.0 M HCl for 2 h at 37° C. followed by neutralization with 55 μl of 2.0 M NaOH. Then, 115 μL of enhancement solution (PerkinElmer) was added to each well and cells were incubated for an additional 30 min at 37° C. prior to reading (see De Silva, et al. Anal Biochem 2010, 398 (1), 15-23). The plates were read on a PerkinElmer VICTORx4 2030 multilabel reader using the standard Eu time-resolved fluorescence (TRF) measurement (340 nm excitation, 400 μs delay, and emission collection for 400 μs at 615 nm). The standard curve was used to determine the amount of ligand present at the Bₘₐₓ obtained in the saturation binding assay. The average number of cells per well at the end of the assay was calculated. To determine the receptor number, the following equation was used: (Eu amount for Bₘₐₓ (mole)/average cell number per well)×6.023×10²³=receptor number per cell. This Bₘₐₓ value was then used to calculate ligand association and binding kinetics for each cell using the following equation: B=B₀+(Bₘₐₓ−B₀)/(1−e^{-kt}), where B corresponds to a receptor saturation parameter, an analog of ligand-receptor complex formation [RL], with values between initial saturation B₀ and the maximum saturation Bₘₐₓ; k is the reaction rate constant, and t is time. The HEK293/SSTR2 cells then were used to assess La³⁺-DOTATAE ligand binding in a europium-based time-resolved fluorescence competitive binding assay as described previously (see Barkey, N. M.; et al. J Med Chem 2011, 54 (23), 8078-84) except that 100 nM of Eu³⁺-DOTATATE was used as the competing ligand. Data points were acquired in quadruplicate and each assay was repeated 3 times.

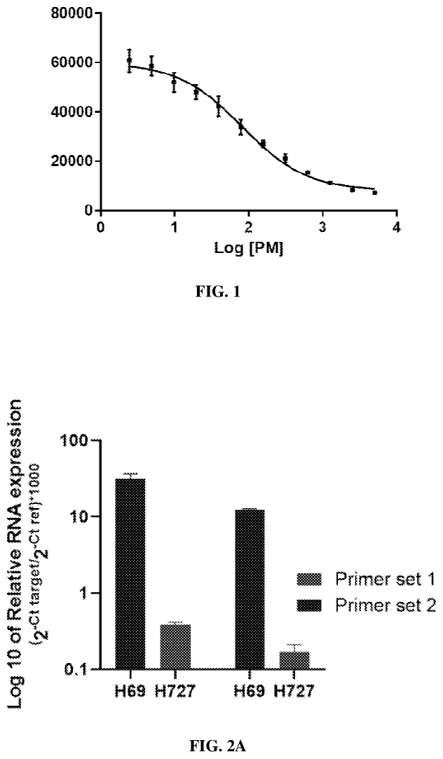

Radiochemical synthesis and characterization. Complexation was achieved by reacting DOTATATE (5-10 μg in 5-10 μL water from 1.0 mg/mL solution) with ²²⁵Ac(NO₃)₃ (3.4 MBq) that was diluted in 100 μL of water containing 10 μL of 20% L-ascorbic acid. The pH was adjusted to 5.5-6 using 1 M Tris buffer (10-12 μL), and then incubated at 60° C. for 1 h (FIG. 1). Reaction progress and radiochemical purity of ²²⁵Ac-DOTATATE were measured without further purification 24 h after collection (to ensure secular equilibrium among ²²⁵Ac and its daughter products) using ITLC with γ counting, radio-TLC, and γ counting of radio-HPLC fractions. In vitro serum stability was determined by adding 50 μL of ²²⁵Ac-DOTATATE (46 μCi) to 1 ml of human serum (n=4), incubated at 37° C. for 10 days and quantified at multiple time points by TLC scanner (Bioscan) and γ-counter (PerkinElmer).
Animal studies. An equal number of male and female subjects were assigned to each experimental cohort. All the animals were purchased from Charles River (Wilmington, MA).
Injected Activity Measurement. Syringes were prefilled with ±10% of ²²⁵Ac-DOTATATE activity. Activities in syringes were measured using the Atomlab 500 Dose Calibrator (Biodex). Per the manufacturer's recommendations, measurements were made for 2 mins using dial number 38.2 and the assumption that ²²⁵Ac and daughters were in secular equilibrium. Activity was injected into each mouse via tail-vein catheter (50 μL dead volume). Residual activities of ²²⁵Ac, and the ²²¹Fr and ²¹³Bi daughter products in catheters and syringes were measured by acquiring isomeric γ spectra immediately after administration using a 4π well-type wipe-test γ counter (BioDex Atomlab 500) post-injection (p.i.) and converted to ²²⁵Ac, ²²¹Fr and ²¹³Bi a activity using factors for γ ray abundance per a decay as described previously (see Tafreshi et al.). The spectra were acquired using a full energy window to include γ counts from ²²⁵Ac, with peak at 99.8 keV (abundance 1%), and its two γ emitting daughters, ²²¹Fr (4.9 min T_{1/2}) with peak at 218.1 keV (abundance 11.4%) and ²¹³Bi (46 min T_{1/2}) with peak at 440.5 keV (abundance 25.9%). The α activity in μCi of each radionuclide was determined by fitting each peak with a multi-Gaussian fit and integrating to determine the net number of counts while incorporating the acquisition time. The net administered activities were determined by subtraction of the residual activity in the catheter and syringe from the dose calibrator measured pre-injection activity. Spectra for activity calculations were acquired ≥24 h post-radiosynthesis ensuring that ²²⁵Ac and daughters were in secular equilibrium (see Robertson, A. K. H.; et al. Phys Med Biol 2017, 62 (11), 4406-4420).
Maximum tolerated activity/toxicity. Cohorts (n=6) of non-tumor bearing BALB/c mice were given a single intravenous injection by tail-vein catheter of a range of ²²⁵Ac-DOTATATE activities (55.5, 111 and 185 kBq in syringes), or saline. Then, mice were weighed twice per week and monitored for 5 months for signs of distressed behavior, followed by euthanasia and tissue collection. Blood urea nitrogen (BUN), creatinine, alkaline phosphatase (ALKP), alanine aminotransferase (ALT) and aspartate aminotransferase (AST) content in serum were analyzed using commercial IDEXX CLIP (IDEXX, Main, USA) and read by an automated biochemical analyzer (IDEXX Catalyst DX, Main, USA) following the manufacturer's protocol. Bone, bone marrow, brain, cecum, duodenum, kidney, liver, lymph nodes, muscle, pancreas, salivary gland, small intestine, and spleen were harvested and fixed in 10% formalin. Bone was decalcified in 14% ethylene diaminetetraacetic acid (EDTA) solution after fixation in formalin. All tissues were embedded in paraffin, sectioned (4-6 μm thickness), stained with hematoxylin and eosin (H&E), and examined by the veterinary pathologist (R.E.), in a blind manner.
Biodistribution (BD). BD studies for ²²⁵Ac-DOTATATE were performed using groups of normal non-tumor bearing BALB/c mice. Each animal received 74 kBq of ²²⁵Ac-DOTATATE a activity in the syringe via tail vein catheter. Groups of animals were euthanized at 24, 48 and 96 h p.i., n=4 mice per time point, and tissues were removed and weighed. Since a particles from ²²⁵Ac and daughters cannot be directly measured in tissue due to the short mean free path, the activities of ²²⁵Ac, ²²¹Fr and ²¹³Bi were measured and converted to a activity as described above for injected activity measurement and previously. Using the net administered activity for ²²⁵Ac, ²²¹Fr and ²¹³Bi, the percent injected activity per gram (% IA/g) were then calculated and compared to a weighed, counted standard for all groups.
Radiation dosimetry. Dosimetry calculations were performed as previously described. As described above for BD studies, isomeric γ-emission spectra of ²²⁵Ac and the two radionuclides in the decay chain with detectable emissions, ²²¹Fr and ²¹³Bi, were acquired in tissues rendered at different p.i. time points and used for these calculations. Because of their short half-lives, ²¹⁷At (32.2 ms) and ²¹³Po (4.2 μs) were assumed to decay at the site of their parent radionuclides, ²²¹Fr and ²¹³Bi respectively. The β decay branching ratio for ²¹⁷At to ²¹⁷Rn is only 0.01% therefore it was assumed that all decays of ²¹⁷At were by a emission to ²¹³Bi. The branching ratios for decay of ²¹³Bi by 98% to ²¹³Po and by 2% to ²⁰⁹T1 were accounted for in the calculation. Due to the relatively low LET and the small dimensions of the target tissues, the β emissions from ²¹⁷At, ²¹³Bi, ²⁰⁹T1 and ²⁰⁹Pb were assumed negligible and not included in the calculations (see Bolch, W. E.; J Nucl Med 2009, 50 (3), 477-84).

Organ and tumor absorbed doses from ²²⁵Ac, ²²¹Fr, ²¹⁷At, ²¹³Bi, and ²¹³Po were determined using acquired BD data. Dosimetry calculations were performed using the generalized internal dosimetry schema of the MIRD Committee for α-particle emitters. According to MIRD #21, the absorbed radiation dose due to particle type x, Dₓ(r_{T}, T_{D}) is the mean energy imparted to target tissue r_{T} per unit tissue mass, defined by Equation 1:

$${D}_{x} ( {r}_{T} , {T}_{D} ) - k \tilde{A} ( {r}_{S} , {T}_{D} ) \frac{{Σ}_{i} {Δ}_{i}^{x} p ( {r}_{T} ← {r}_{S} ; {E}_{i}^{x} )}{M ( {r}_{T} )} \qquad Eq . 1$$

where A(r_{S}, T_{D}) is the total number of nuclear transitions in the target region (accumulated activity), Δᵢ^{x} is the mean energy emitted per nuclear transition, φ(r_{T}←r_{S}; Eᵢ^{x}) is the fraction of energy emitted per nuclear transition in the source region that is absorbed in the target region by the i^{th} emission that is emitted with initial energy E, k is a conversion factor, and M(r_{T}) is the mass of the target tissue. Time-activity curves were generated for each organ and fit with an exponential decay nonlinear regression. Accumulated activity in each organ/tumor was determined by analytically integrating the resulting equation of fit. Several assumptions were made in the calculation of absorbed dose. It was assumed that the α-particle activity was uniformly distributed in each organ/tumor volume and as a result of its short range in tissue, no a particles escaped from its source organ/tumor. Electron and photon contributions were assumed to be negligible compared to α-particle energy deposition (see Kratochwil, C.; et al. J Nucl Med 2017). After these assumptions, Equation 1 simplifies to:

$${D}_{a} ( r , {T}_{n} ) = \frac{k \tilde{A} ( r , {T}_{D} ) {Λ}^{a}}{M ( r )} \qquad Eq . 2$$

It was assumed that a particles from ²²¹Fr (4.9 min T_{1/2}), ²¹⁷At (32.2 ms T_{1/2}), ²¹³Bi (46 min T_{1/2}) and ²¹³Po (4.2 μs T_{1/2}) were deposited in the same location as ²²⁵Ac (10 d T_{1/2}) due to the relatively shorter half-lives of these daughter isotopes. Although ²¹⁷At and ²¹³Po do not have detectable γ emissions, under the assumption that the decay chain had reached secular equilibrium, the accumulated activity of these two daughters would equal that of ²²¹Fr and ²¹³Bi, respectively. The total absorbed α-particle dose was calculated from the summation of doses from ²²⁵Ac, ²²¹Fr, ²¹⁷At, ²¹³Bi and ²¹³Po.
Anti-tumor efficacy. Efficacy studies were performed using NCI-H69 human SCLC and NCI-H727 lung carcinoid xenograft tumors in SCID mice. The tumor bearing mice (n=10/group) were injected with quantified activities of either ²²⁵Ac-DOTATATE or saline solution (0.9%, pharmaceutical grade, Cardinal Pharmaceuticals, OH). Animals were euthanized when the experimental endpoint of 2000 mm³ tumor volume was reached. Any euthanasia performed prior to reaching 2000 mm³ tumor volume were due to clinical endpoints, such as 20% weight loss, tumor ulceration, hunched back, scruffy fur (no longer grooming), lethargic or slow moving.
Histology and immunohistochemistry (IHC). Following euthanasia, organs (for toxicity study) and tumors (for efficacy study) were excised, fixed in formalin, embedded in paraffin, sectioned (5 μm), and stained with H&E (for both tumors and organs) and IHC (for tumors). IHC staining was performed using rabbit SSTR2 polyclonal antibody (1:200 dilution, GTX70735, GeneTex) and a Discovery XT automated system (Ventana Medical Systems, Tucson, AZ). Slides were scanned using a ScanScope XT digital slide scanner (Aperio, CA). To quantify SSTR2 expression in tumors, images from serial H&E and IHC central sections were analyzed using Visiopharm 2017.7. Each serial section pair (H&E and IHC) were aligned using the tissue align module and viable tumor segmented by thresholding the hematoxylin channel. A multi-threshold marker area analysis was then performed within the viable tumor region on each IHC image. Each pixel was placed into 4 categories (Negative, Weak, Moderate, Strong) based on thresholds set by a pathologist and percentages of each category were normalized by total area of interest.
Statistical analysis. Cohort size was determined by power analyses. Except for efficacy studies, subjects were randomly assigned to experimental cohorts. Due to concern that variance in mean tumor volumes among the experimental cohorts could adversely influence results, subjects in efficacy studies were assigned to cohorts such that the distribution of tumor volumes and mean tumor volumes were consistent among the groupings. Pathologists were blinded to the experimental cohorts. The toxicity data of ²²⁵Ac-DOTATATE was investigated for three different dose levels as compared to control using weight gain, CREA, BUN, ALKP, AST and ALT. An analysis of variance (ANOVA) was used to assess whether there was a difference outcome in the dose levels overall and Dunnett's test to see whether there is a difference between each dose level and control (saline). Prior to performing the analysis of variance, the Anderson Darling test was used to assess whether a transformation was necessary. For the efficacy studies, two-way ANOVA was used to compare tumor size among the treatment and saline groups and Kaplan-Meier analyses (GraphPad Prism 7 software) were used to determine significance among the cohorts.

### Results

Binding affinity of La³⁺-DOTATATE. Since there are no non-radioactive isotopes of Ac, the analogous La³⁺-DOTATATE chelate was prepared to determine its affinity. La is a useful surrogate for compounds being developed with Ac, since both exist as trivalent ions in solution (see Morss, L. R.; et al. The Chemistry of the Actinide and Transactinide Elements, Volumes 1-6. 4th ed.; Springer Science and Business Media: 2011). The HRMS (ESI) calculated mass for La³⁺-DOTATATE, C₆₅H₈₇LaN₁₄O₁₉S₂ (M+H)⁺, 1571.4855 MW, was found to be 1571.4867. HPLC purity was 100%, t_{R}=22.15 min. The La³⁺-DOTATATE chelate was determined to bind SSTR2 expressing HEK293/SSTR2 cells with high, 19.00±9.2 nM Ki, affinity as shown by time-resolved fluorescence (TRF) competitive binding assay (FIG. 1).

HEK/293 cells and Eu³⁺-DOTATATE were used for the whole-cell TRF saturation binding assay to determine binding and cell-surface receptor number. The HRMS (ESI) calculated mass for the Eu³⁺-DOTATATE chelate, C₆₅H₈₇EuN₁₄O₁₉S₂(M+H)⁺, 1585.5004 MW, was found to be 1585.5031. HPLC purity was 100%, t_{R}=22.14 min. Eu³⁺-DOTATATE binding affinity for SSTR2 was determined to be 22.12 nM K_{d}, and it was determined that, on average, HEK293/SSTR2 cells express 7.3×10⁵ SSTR2 receptors on the cell-surface per cell by TRF saturation binding assay.

Radiosynthesis and characterization of ²²³Ac-DOTATATE. Radiosynthesis (Scheme 1) provided a greater than 98% yield with high radiochemical purity (≥99.8%), as determined by radio-TLC, γ-counter quantification, and radio-HPLC. Also, an excellent in vitro stability of the ²²⁵Ac-DOTATATE was demonstrated, i.e., 97% integrity remaining after 2 days in human serum at 37° C. (Table 1).

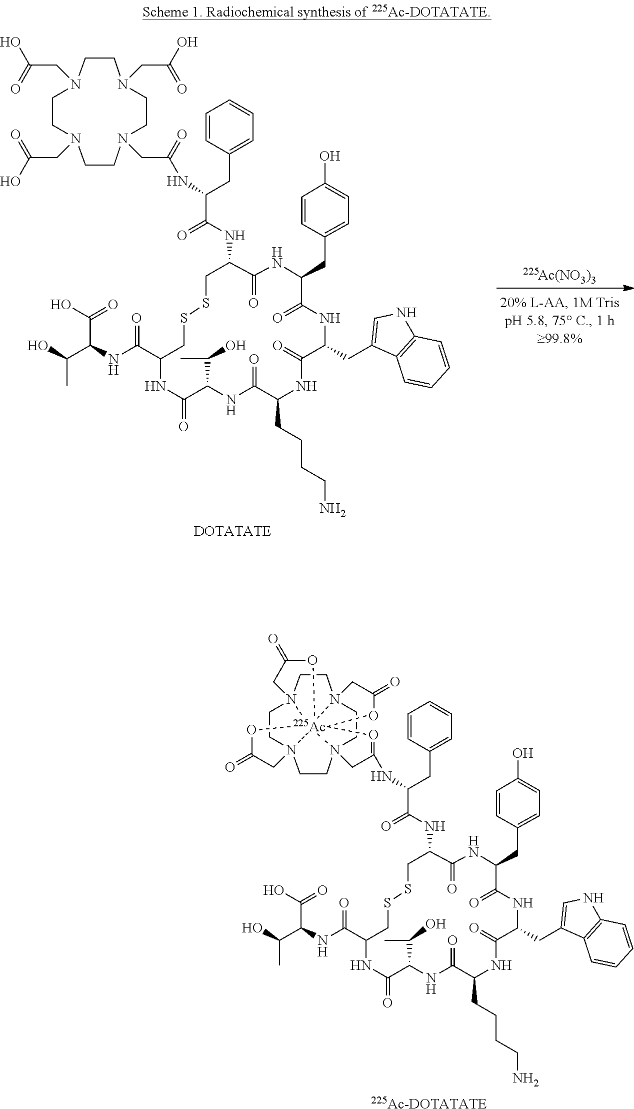

TABLE 1
In vitro serum stability of ²²⁵Ac-DOTATATE.

| | % Intact |
|---|---|
| Day | TLC scanner |
| 0 | 100 |
| 1 | 100.44 ± 0.88 |
| 3 | 99.27 ± 0.29 |
| 5 | 96.74 ± 2.06 |
| 7 | 93.19 ± 1.50 |
| 10 | 90.29 ± 4.00 |

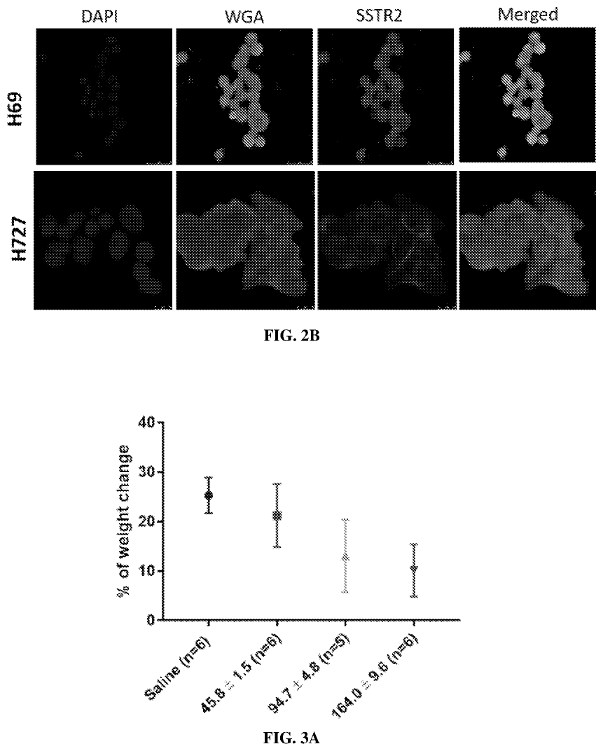

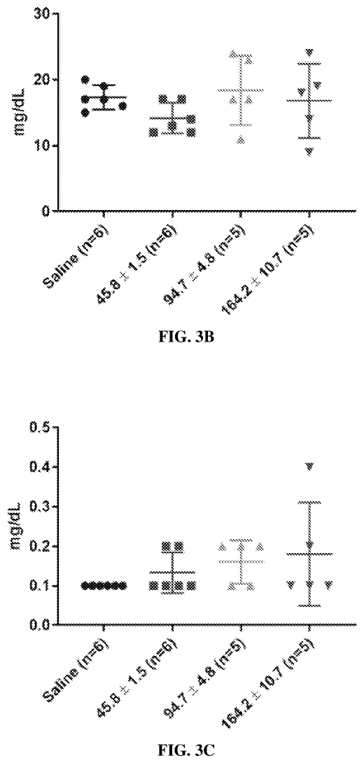

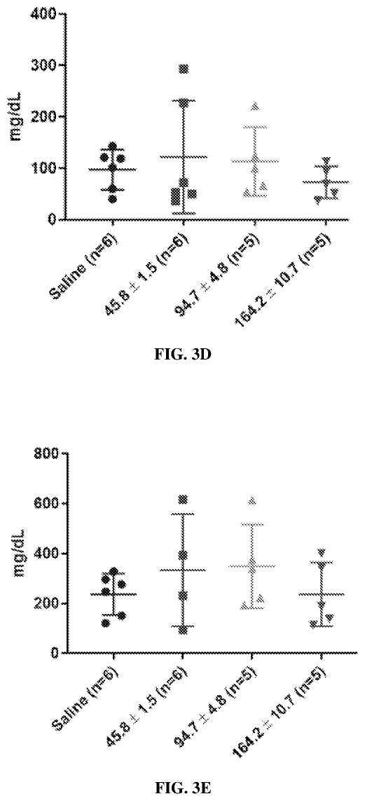

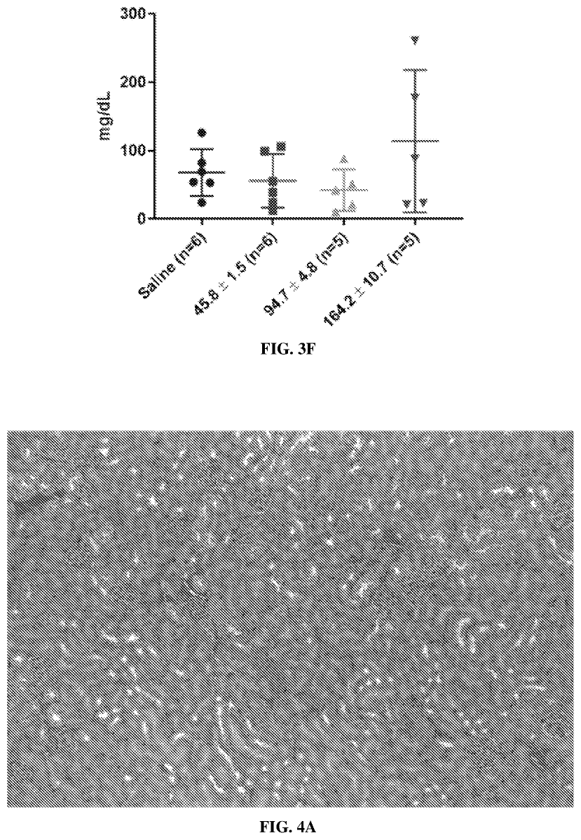

SSTR2 expression in H69 and H727 cells. SSTR2 expression was verified in H69 and H727 cell lines by qRT-PCR and ICC. H69 cells have significantly higher mRNA (p<0.001) and protein expression (p<0.05) relative to H727 cells (FIGS. 2A-2B).
Toxicity. The toxicity of ²²⁵Ac-DOTATATE was evaluated in immune-competent non-tumor bearing BALB/c mice (n=6/cohort). Cohorts received a single i.v. injection of ²²⁵Ac-DOTATATE over the range of 0-185 kBq in the syringe. At completion of the study (5 months p.i.) serum and tissues were collected. Tissues were sectioned and H& E stained for pathology examination to determine activity associated damage. Blood urea nitrogen (BUN) and creatinine, which are important indicators of renal function, and ALP, AST, and ALKP, which are important indicators of liver function were determined. Weight gain was observed in all animals by the end of the study, albeit less weight was gained by animals at the highest dose level relative to the lowest (FIG. 3A). There are significant differences in weight change between saline and 111 kBq and 185 kBq (p<0.01 and p<0.001, respectively), but not 55.5 kBq (p=0.50). Blood BUN and creatinine levels were all in the normal range (FIGS. 3B-3C) with p-values=0.12 and 0.33, respectively, indicating that there is no significant difference in BUN and creatinine among the cohorts with injected activity relative to the saline control. ANOVA and Dunnett's test determined that no significant differences were observed among the cohorts in ALT, AST or ALKP liver enzymes (FIGS. 3D-3F).

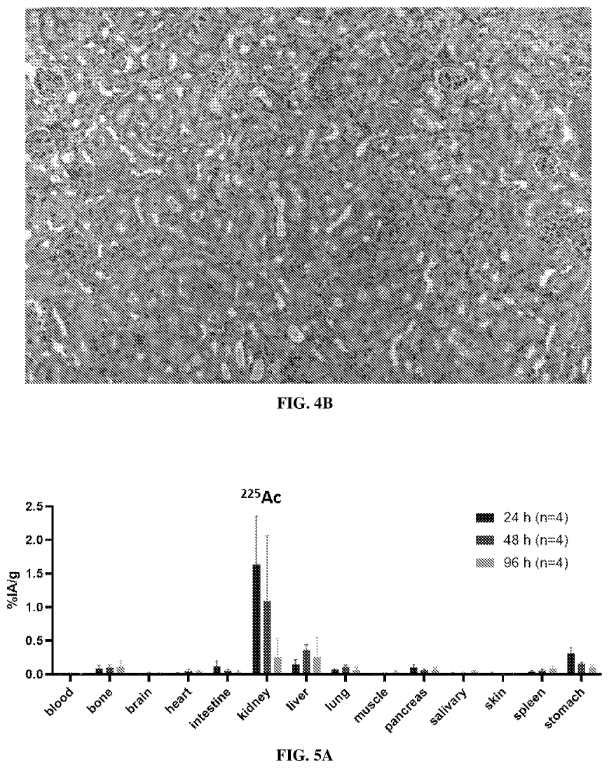

Histopathologic analysis showed no pathologic findings after injection of 55.5 and 111 kBq in the syringe and these activities were well tolerated by the mice. In contrast, after injection of higher activities of ≥111 kBq in the syringe (≥94.8 kBq actual injected activity) chronic progressive nephropathy was observed (FIGS. 4A-4B). These groups of the animals also started losing weight around 100 days p.i. None of the other studied organs (Bone, bone marrow, brain, cecum, duodenum, liver, lymph nodes, muscle, pancreas, salivary gland, small intestine, and spleen) showed pathologic changes related to the treatment. Glycogen accumulation was observed in the livers of some animals (including animals in the saline control group), but this was considered an incidental finding.

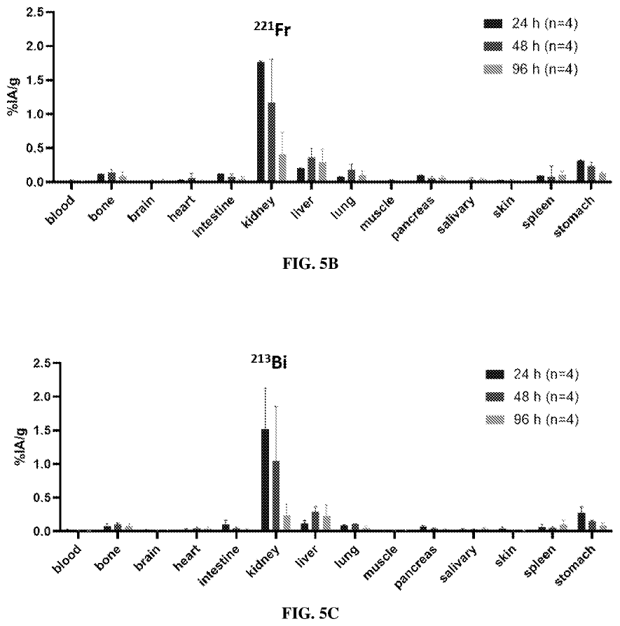

Biodistribution. To evaluate the biodistribution (BD) of ²²⁵Ac-DOTATATE, non-tumor bearing BALB/c mice (n=4 per cohort) were injected intravenously with 74 kBq of ²²⁵Ac-DOTATATE a activity in the syringe. As described in the Methods, the α-particle activities from ²²⁵Ac and daughters ²²¹Fr and ²¹³Bi were determined for each organ and time-point using γ spectroscopy (FIGS. 5A-5C). At 24 hours p.i. the kidneys, liver and stomach had 1.63±0.72, 0.147±0.07 and 0.31±0.08% IA/g, respectively, while only negligible activity was observed in the other tissues measured. Activity had largely cleared from the tissues by 96 h p.i.
Radiation Dosimetry. Radiation dosimetry (RD) calculations were based on the data obtained from the BD studies at 24, 48 and 96 h p.i. RD for targeted radiotherapy is the determination of the absorbed energy deposited per unit mass by ionizing radiation in the different tissue compartments within the body. As described in the Methods, the α-particle dose from ²²⁵Ac and each of its α-emitting daughters was calculated using γ spectroscopy of ²²⁵Ac, ²²¹Fr and ²¹³Bi. The decay of ²²¹Fr to ²¹⁷At was assumed to take place in the same location as ²²¹Fr. Similarly, the decay of ²¹³Po was assumed to take place at the same location as ²¹³Bi, while accounting for its 98% branching ratio.

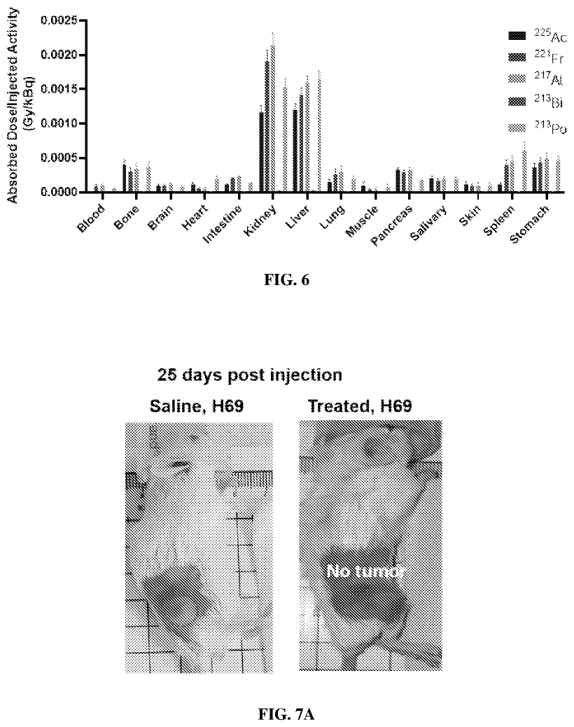

BD data for the different tissues were fitted using an exponential decay nonlinear regression, allowing the estimation of clearance kinetics, tissue biological half-life, accumulated activity, and absorbed dose/injected activity (Gy/kBq) for each radionuclide in each tissue (Table 2). The total absorbed dose is the summation of the values for the five α-emitting radionuclides. The calculated total absorbed dose for ²²⁵Ac-DOTATATE was minimal in all tissues except clearance organs. The total absorbed doses of ²²⁵Ac and daughters were greatest in the kidney and liver, 0.0068 and 0.0059 Gy/kBq, respectively. FIG. 6 represents graphs of the absorbed dose from each radionuclide per tissue.

It is noted that the effective decay half-lives (T_{eff}) calculated for ²²⁵Ac in tissues that had significant uptake were shorter than the radiodecay half-life of ²²⁵Ac (10 d), indicating biological clearance. For example, the calculated T_{eff} in liver and kidney was 1.15 and 5.40 days, respectively (Table 2). Hence, T_{eff} is a composite of radiodecay and active biological clearance. The T_{eff} was only calculated to be longer in some tissues with minimal uptake where instrument background likely interfered with the accuracy of measurement.

TABLE 2
Radiation dosimetry and clearance kinetics parameters
for ²²⁵Ac-DOTATATE in BALB/c mice.

²²⁵Ac

| Parameter | Blood | Bone | Brain | Heart | Intestine | Kidney | Liver |
|---|---|---|---|---|---|---|---|
| Initial activity/organ, | 0.0003 | 0.0131 | 0.0019 | 0.0033 | 0.1527 | 0.3862 | 0.1200 |
| A₀ (kBq) | | | | | | | |
| Decay rate constant, λ_{eff} | 0.0206 | 0.0030 | −0.0103 | −0.0004 | 0.0200 | 0.0250 | −0.0053 |
| (h⁻¹) | | | | | | | |
| Decay half-life, T_{eff} (days) | 1.4020 | 9.6270 | 2.8040 | 72.2028 | 1.4441 | 1.1552 | 5.4493 |
| Accumulated | 0.0080 | 1.4274 | 0.8190 | 0.4871 | 4.4589 | 8.2462 | 29.4458 |
| activity/organ, Ã (kBq*h) | | | | | | | |
| Absorbed | 0.0000 | 0.0004 | 0.0001 | 0.0001 | 0.0001 | 0.0012 | 0.0012 |
| dose/injected | | | | | | | |
| activity (Gy/kBq) | | | | | | | |

²²⁵Ac

| Parameter | Lung | Muscle | Pancreas | Salivary | Skin | Spleen | Stomach |
|---|---|---|---|---|---|---|---|
| Initial activity/organ, | 0.0227 | 0.0024 | 0.0171 | 0.0028 | 0.0023 | 0.0039 | 0.0859 |
| A₀ (kBq) | | | | | | | |
| Decay rate constant, λ_{eff} | 0.0130 | −0.0049 | 0.0060 | −0.0077 | −0.0092 | 0.0062 | 0.0190 |
| (h⁻¹) | | | | | | | |
| Decay half-life, T_{eff} (days) | 2.2216 | 5.8941 | 4.8135 | 3.7508 | 3.1393 | 4.6582 | 1.5201 |
| Accumulated | 1.0829 | 0.5613 | 1.4271 | 0.8906 | 0.8656 | 0.3328 | 2.6801 |
| activity/organ, Ã (kBq*h) | | | | | | | |
| Absorbed | 0.0001 | 0.0001 | 0.0003 | 0.0002 | 0.0001 | 0.0001 | 0.0004 |
| dose/injected | | | | | | | |
| activity (Gy/kBq) | | | | | | | |

²²¹Fr

| Parameter | Blood | Bone | Brain | Heart | Intestine | Kidney | Liver |
|---|---|---|---|---|---|---|---|
| Initial activity/organ, | 0.0019 | 0.0170 | 0.0036 | 0.0067 | 0.1532 | 0.4011 | 0.1612 |
| A₀ (kBq) | | | | | | | |
| Decay rate constant, λ_{eff} | −0.0071 | 0.0100 | −0.0044 | 0.0190 | 0.0130 | 0.0190 | −0.0033 |
| (h⁻¹) | | | | | | | |
| Decay half-life, T_{eff} (days) | 4.0678 | 2.8881 | 6.5639 | 1.5201 | 2.2216 | 1.5201 | 8.7519 |
| Accumulated | 0.5594 | 1.0195 | 0.7934 | 0.2102 | 7.2991 | 12.5136 | 32.1637 |
| activity/organ, Ã (kBq*h) | | | | | | | |
| Absorbed | 0.0001 | 0.0003 | 0.0001 | 0.0000 | 0.0002 | 0.0019 | 0.0014 |
| dose/injected | | | | | | | |
| activity (Gy/kBq) | | | | | | | |

²²¹Fr

| Parameter | Lung | Muscle | Pancreas | Salivary | Skin | Spleen | Stomach |
|---|---|---|---|---|---|---|---|
| Initial activity/organ, | 0.0251 | 0.0052 | 0.0169 | 0.0037 | 0.0039 | 0.0097 | 0.0852 |
| A₀ (kBq) | | | | | | | |
| Decay rate constant, λ_{eff} | 0.0080 | 0.0170 | 0.0080 | −0.0028 | −0.0015 | 0.0030 | 0.0170 |
| (h⁻¹) | | | | | | | |
| Decay half-life, T_{eff} (days) | 3.6101 | 1.6989 | 3.6101 | 10.3147 | 19.2541 | 9.6270 | 1.6989 |
| Accumulated | 1.7734 | 0.1848 | 1.1933 | 0.7036 | 0.6489 | 1.0604 | 3.0452 |
| activity/organ, Ã (kBq*h) | | | | | | | |
| Absorbed | 0.0003 | 0.0000 | 0.0003 | 0.0002 | 0.0001 | 0.0004 | 0.0004 |
| dose/injected | | | | | | | |
| activity (Gy/kBq) | | | | | | | |

²¹⁷At

| Parameter | Blood | Bone | Brain | Heart | Intestine | Kidney | Liver |
|---|---|---|---|---|---|---|---|
| Initial activity/organ, | 0.0019 | 0.0170 | 0.0036 | 0.0067 | 0.1532 | 0.4011 | 0.1612 |
| A₀ (kBq) | | | | | | | |
| Decay rate constant, λ_{eff} | −0.0071 | 0.0100 | −0.0044 | 0.0190 | 0.0130 | 0.0190 | −0.0033 |
| (h⁻¹) | | | | | | | |
| Decay half-life, T_{eff} (days) | 4.0678 | 2.8881 | 6.5639 | 1.5201 | 2.2216 | 1.5201 | 8.7519 |
| Accumulated | 0.5594 | 1.0195 | 0.7934 | 0.2102 | 7.2291 | 12.5136 | 32.1627 |
| activity/organ, Ã (kBq*h) | | | | | | | |
| Absorbed | 0.0001 | 0.0003 | 0.0001 | 0.0001 | 0.0002 | 0.0021 | 0.0016 |
| dose/injected | | | | | | | |
| activity (Gy/kBq) | | | | | | | |

²¹⁷At

| Parameter | Lung | Muscle | Pancreas | Salivary | Skin | Spleen | Stomach |
|---|---|---|---|---|---|---|---|
| Initial activity/organ, | 0.0251 | 0.0052 | 0.0169 | 0.0037 | 0.0039 | 0.0097 | 0.0852 |
| A₀ (kBq) | | | | | | | |
| Decay rate constant, λ_{eff} | 0.0080 | 0.0170 | 0.0080 | −0.0028 | −0.0015 | 0.0030 | 0.0170 |
| (h⁻¹) | | | | | | | |
| Decay half-life, T_{eff} (days) | 3.6101 | 1.6989 | 3.6101 | 10.3147 | 19.2541 | 9.6270 | 1.6989 |
| Accumulated | 1.7734 | 0.1848 | 1.1933 | 0.7036 | 0.6489 | 1.0604 | 3.0452 |
| activity/organ, Ã (kBq*h) | | | | | | | |
| Absorbed | 0.0003 | 0.0000 | 0.0003 | 0.0002 | 0.0001 | 0.0004 | 0.0005 |
| dose/injected | | | | | | | |
| activity (Gy/kBq) | | | | | | | |

²¹³Bi

| Parameter | Blood | Bone | Brain | Heart | Intestine | Kidney | Liver |
|---|---|---|---|---|---|---|---|
| Initial activity/organ, | 0.0042 | 0.0125 | 0.0053 | 0.1340 | 0.3639 | 0.0948 | 0.0270 |
| A₀ (kBq) | | | | | | | |
| Decay rate constant, λ_{eff} | 0.0110 | 0.0070 | 0.0080 | 0.0030 | 0.0210 | 0.0250 | −0.0073 |
| (h⁻¹) | | | | | | | |
| Decay half-life, T_{eff} (days) | 2.6256 | 4.1259 | 3.6101 | 9.6270 | 1.3753 | 1.1552 | 3.9563 |
| Accumulated | 0.2345 | 0.9591 | 0.4653 | 0.5789 | 3.6681 | 7.7700 | 28.7854 |
| activity/organ, Ã (kBq*h) | | | | | | | |
| Absorbed | 0.0000 | 0.0000 | 0.0000 | 0.0000 | 0.0000 | 0.0000 | 0.0000 |
| dose/injected | | | | | | | |
| activity (Gy/kBq) | | | | | | | |

²¹³Bi

| Parameter | Lung | Muscle | Pancreas | Salivary | Skin | Spleen | Stomach |
|---|---|---|---|---|---|---|---|
| Initial activity/organ, | 0.0270 | 0.0034 | 0.0119 | 0.0056 | 0.0056 | 0.0068 | 0.0777 |
| A₀ (kBq) | | | | | | | |
| Decay rate constant, λ_{eff} | 0.0170 | 0.0050 | 0.0140 | 0.0030 | 0.0040 | −0.0024 | 0.0190 |
| (h⁻¹) | | | | | | | |
| Decay half-life, T_{eff} (days) | 1.6989 | 5.7762 | 2.0629 | 9.6270 | 7.2203 | 12.0338 | 1.5201 |
| Accumulated | 0.9636 | 0.3058 | 0.5253 | 0.6047 | 0.5575 | 1.2470 | 2.4230 |
| activity/organ, Ã (kBq*h) | | | | | | | |
| Absorbed | 0.0000 | 0.0000 | 0.0000 | 0.0000 | 0.0000 | 0.0000 | 0.0000 |
| dose/injected | | | | | | | |
| activity (Gy/kBq) | | | | | | | |

²¹³Po

| Parameter | Blood | Bone | Brain | Heart | Intestine | Kidney | Liver |
|---|---|---|---|---|---|---|---|
| Initial activity/organ, | 0.0042 | 0.0125 | 0.0066 | 0.0053 | 0.1340 | 0.3639 | 0.0948 |
| A₀ (kBq) | | | | | | | |
| Decay rate constant, λ_{eff} | 0.0110 | 0.0070 | 0.0080 | 0.0030 | 0.0210 | 0.0250 | −0.0073 |
| (h⁻¹) | | | | | | | |
| Decay half-life, T_{eff} (days) | 2.6256 | 4.1259 | 3.6101 | 9.6270 | 1.3753 | 1.1552 | 3.9563 |
| Accumulated | 0.2345 | 0.9591 | 0.4653 | 0.5789 | 3.6681 | 7.7700 | 28.7854 |
| activity/organ, Ã (kBq*h) | | | | | | | |
| Absorbed | 0.0000 | 0.0004 | 0.0001 | 0.0002 | 0.0001 | 0.0015 | 0.0016 |
| dose/injected | | | | | | | |
| activity (Gy/kBq) | | | | | | | |

²¹³Po

| Parameter | Lung | Muscle | Pancreas | Salivary | Skin | Spleen | Stomach |
|---|---|---|---|---|---|---|---|
| Initial activity/organ, | 0.0270 | 0.0034 | 0.0119 | 0.0056 | 0.0056 | 0.0068 | 0.0777 |
| A₀ (kBq) | | | | | | | |
| Decay rate constant, λ_{eff} | 0.0170 | 0.0050 | 0.0140 | 0.0030 | 0.0040 | −0.0024 | 0.0190 |
| (h⁻¹) | | | | | | | |
| Decay half-life, T_{eff} (days) | 1.6989 | 5.7762 | 2.0629 | 9.6270 | 7.2203 | 12.0338 | 1.5201 |
| Accumulated | 0.9636 | 0.3058 | 0.5253 | 0.6047 | 0.5575 | 1.2470 | 2.4230 |
| activity/organ, Ã (kBq*h) | | | | | | | |
| Absorbed | 0.0002 | 0.0001 | 0.0002 | 0.0002 | 0.0001 | 0.0006 | 0.0005 |
| dose/injected | | | | | | | |
| activity (Gy/kBq) | | | | | | | |

| | Blood | Bone | Brain | Heart | Intestine | Kidney | Liver |
|---|---|---|---|---|---|---|---|
| Total absorbed | 0.0002 | 0.0014 | 0.0004 | 0.0004 | 0.0007 | 0.0068 | 0.0059 |
| dose/injected | | | | | | | |
| Activity (Gy/kBq) | | | | | | | |

| | Lung | Muscle | Pancreas | Salivary | Skin | Spleen | Stomach |
|---|---|---|---|---|---|---|---|
| Total absorbed | 0.0009 | 0.0003 | 0.0011 | 0.0008 | 0.0004 | 0.0016 | 0.0017 |
| dose/injected | | | | | | | |
| Activity (Gy/kBq) | | | | | | | |

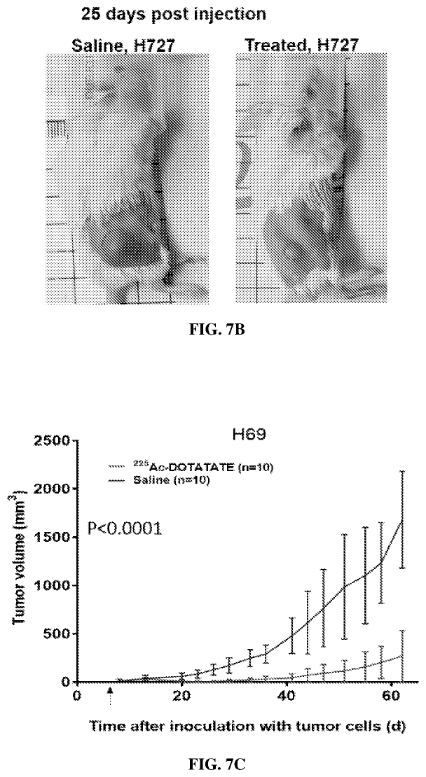

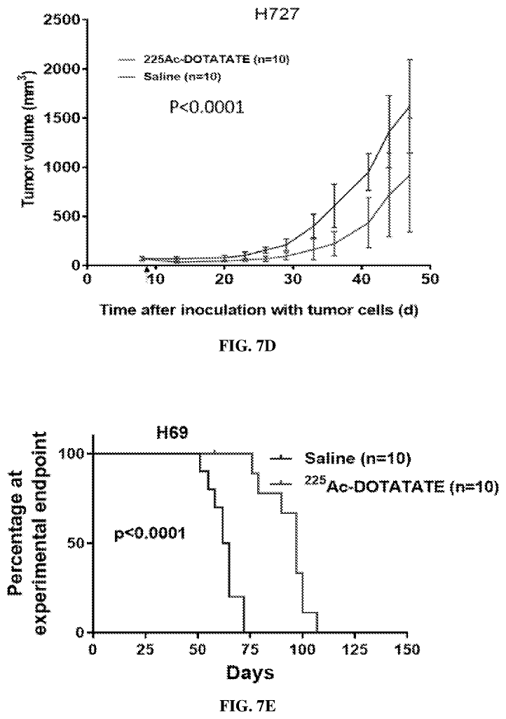

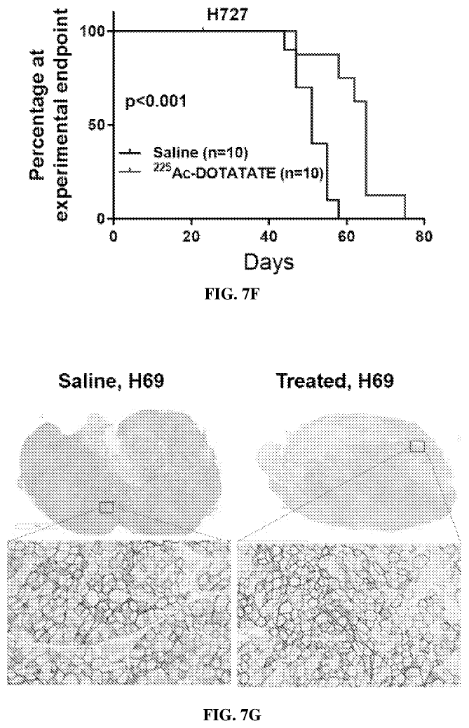

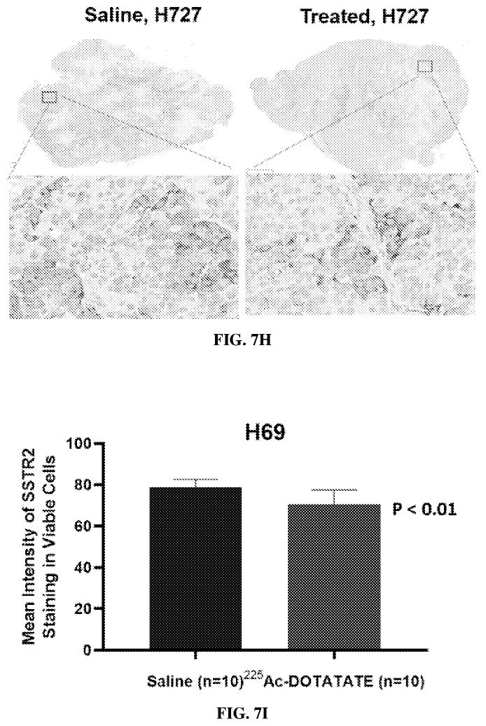

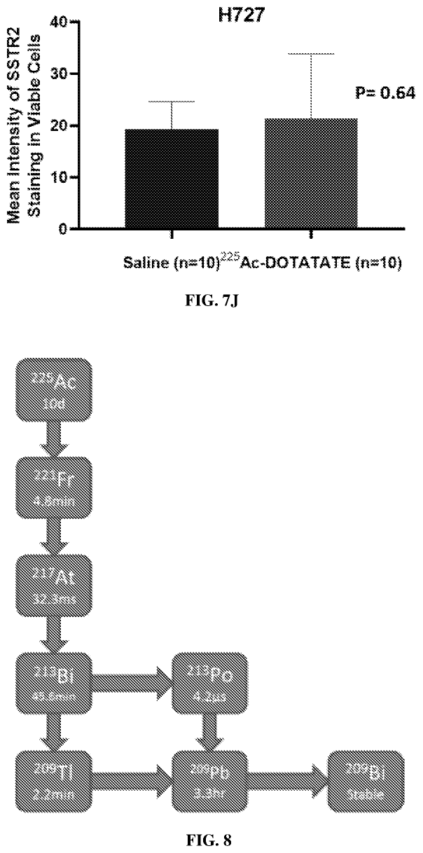

Anti-tumor Efficacy. Two subcutaneous xenograft tumor models were used to assess the anti-tumor effect of ²²⁵Ac-DOTATATE in vivo, H69 human SCLC and H727 human lung carcinoid. As reported above, the H69 and H727 tumor cell lines have high and low endogenous expression of SSTR2, respectively. Eight days after tumor cell xenoengraftment, mice (n=10 per cohort) were intravenously injected with a single bolus of 148.7 kBq or 144.3 kBq mean ²²⁵Ac activity for H69 and H727 cohorts, respectively, or saline solution. Animal weights and tumor volumes were measured twice weekly (starting 5 days p.i.) until the tumors reached the experimental endpoint of 2000 mm³. FIGS. 7A-7B show representative images at 25 days p.i. (33 days post tumor cell xenoengraftment). Tumor volumes decreased significantly after treatment relative to saline controls (p<0.001) prior to eventual regrowth (FIGS. 7C-7D). Saline control mice had significantly greater tumor volumes at the 25 d p.i. time point (p<0.0001) compared to mice treated with ²²⁵Ac-DOTATATE. FIGS. 7E-7F show Kaplan-Meier analyses. Animals treated with ²²⁵Ac-DOTATATE had a significantly delayed time to experimental endpoint (p<0.0001 for H69 and p=0.0009 for H727) relative to the saline controls. The median time to endpoint was 93.6±10.1 and 62.7±6.7 days for the H69 treated and saline cohorts, respectively, and the median survival was 62.7±7.9 days and 51.4±4.4 for the H727 treated and saline cohorts, respectively.

To determine if a single bolus administration of ²²⁵Ac-DOTATATE selected for decreased target expression, tumor sections were IHC stained for SSTR2 expression after reaching the experimental endpoint of 2000 mm³ tumor volume (FIGS. 7G-7H) and SSTR2 staining was quantified (FIGS. 7I-7J). While the level of SSTR2 expression was not significantly different in treated H727 tumors relative to saline controls (p=0.64), H69 treated tumors had 10% lower expression relative to controls (p<0.01).

### DISCUSSION

Clinically, TAT is an exciting and promising therapeutic modality for SSTR2-expressing NETs. TAT has potential advantages over TBT due to the high linear energy transfer and short path length of a particles, which can cause relatively greater DNA double-stranded breaks in a smaller volume when compared to β particles. Further, α particles have demonstrated greater cell-death in hypoxic regions of tumors relative to β. Since SSTR2 receptors have heterogeneity of expression in carcinoid tumors and metastases, TAT is potentially ideal as the energy release encompasses a volume of multiple cell diameters, allowing for killing of adjacent cells that do not express the target receptor. Whereas, β emissions have a range of millimeters, potentially extending beyond the tumor boundary, with the greatest proportion of energy deposition occurring at the extent of the range, causing toxicity to surrounding normal tissues. In demonstration of the advantages of a particles to β particles, SSR TAT has been compared with SSR TBT in several studies and TAT demonstrated long-lasting efficacy in pre-clinical and clinical neuroendocrine tumors with resistance to TBT.

The α-emitting generator radionuclide ²²⁵Ac has been proposed to be a potent cytotoxic agent for NET TAT. A variety of ²²⁵Ac-nanogenerator targeted therapies have been developed that concentrate a emissions in the decay-chain within the target tumors, with limited translocation of daughter isotopes (see Miederer, M.; et al. Adv Drug Deliv Rev 2008, 60 (12), 1371-82). In this example, the BD, RD, toxicity, and therapeutic efficacy of ²²⁵Ac-DOTATATE in preclinical models of somatostatin receptor 2 (SSTR2) positive lung tumors has been evaluated. DOTATATE was chosen in this study because ¹⁷⁷Lu-DOTATATE has been compared with ¹⁷⁷Lu-DOTATOC in patients and a greater tumor residence time was observed for ¹⁷⁷Lu-DOTATATE, indicating that DOTATATE may be a superior targeting ligand for delivery of radiotherapy (see Esser, J. P.; et al. Eur J Nucl Med Mol Imaging 2006, 33 (11), 1346-51). Herein, a high affinity of La³⁺-DOTATATE was demonstrated for SSTR2, where La³⁺ is an established non-radioactive surrogate for ²²⁵Ac due to their chemical similarity. High radiochemical yield, purity and stability in plasma were demonstrated for ²²⁵Ac-DOTATATE.

Herein, the first preclinical animal study for ²²⁵Ac-DOTATATE is reported. Ballal, et al., recently presented the first clinical experience and early results on the efficacy and safety of ²²⁵Ac-DOTATATE in 32 patients with SSTR expressing metastatic GEP-NETs who were, ¹⁷⁷Lu-DOTATATE refractory, as the last-line therapy option after exhausting all the standard available therapies. Systemic TAT using ²²⁵Ac-DOTATATE was performed in all the patients with ²²⁵Ac-DOTATATE (100 kBq/kg body weight) at an interval of 8 weeks, to a cumulative dose of 55,500 kBq (1.5 mCi). This pilot study was reported at an early stage, too soon to calculate overall and event-free survival. However, the short-term clinical results with a median follow-up duration of 8 months suggests that treatment with ²²⁵Ac-DOTATATE can overcome resistance to ¹⁷⁷Lu-DOTATATE and that ²²⁵Ac-DOTATATE can be used as a promising treatment option. Despite this early clinical experience, preclinical animal studies are needed to better understand the biodistribution and radiation dosimetry of ²²⁵Ac-DOTATATE, and to support the development of novel methods of determining radiation dosimetry in patients.

Previous somatostatin receptor-targeted PRRT studies have shown that nephrotoxicity is a dose-limiting factor since reabsorption of radio-labelled SSAs by cells in the proximal tubule of the nephron occurs (see Lambert, B.; et al. Radiat Res 2004, 161 (5), 607-11; and Vegt, E.; et al. J Nucl Med 2010, 51 (7), 1049-58). Apreclinical study on PRRT with ²²⁵Ac-DOTATOC conducted in nude mice bearing AR42J rat pancreatic NET xenograft tumors demonstrated significant kidney tubular necrosis at activities higher than 30 kBq. The BD results herein demonstrated that ²²⁵Ac-DOTATATE is primarily cleared by the renal route, with some hepatic clearance. Clearance kinetics parameters and RD were calculated, and the highest accumulated doses were observed in the kidneys and liver. In the toxicity study, all animals survived to 5-months p.i. and there was no acute kidney damage observed by pathology at any dose level. BUN and creatinine levels were in the normal range for all animals in the study. However, chronic progressive nephropathy was observed in animals injected with ≥2.56 μCi (94.72 kBq) and, despite overall weight gain, animals that were injected with the highest activities, averaging 4.43 μCi, 164 kBq, began losing weight at ˜100-days p.i. It is notable that for clinical administrations, SSA PRRTs are co-infused with amino acids for renal protection by blocking peptide reabsorption in the proximal tubules, which significantly diminishes the radiation dose to the kidneys (see Rolleman, E. J.; et al. Eur J Nucl Med Mol Imaging 2003, 30 (1), 9-15). Liver enzymes were normal and there was no treatment related damage observed in the liver or other tissues by pathology.

A high and moderate endogenous expression of SSTR2 in H69 and H727 human lung cancer cells, respectively, was confirmed. Following a single treatment of ²²⁵Ac-DOTATATE, the in vivo efficacy study demonstrated significantly decreased tumor volume, increased tumor growth delay and prolonged time to experimental endpoint for animals bearing both tumor types. However, the therapeutic effects were significantly greater in H69 tumor-bearing animals compared to H727 tumor bearing animals, and this is likely due to the relatively higher target expression in H69 tumors. It is notable that H69 tumors that regrew following treatment had 10% lower SSTR2 target expression relative to controls and this difference was significant, but the H727 tumors re-grew with unaltered expression.

In conclusion, ²²⁵Ac-DOTATATE was synthesized with high radiochemical purity and yield and performed stability, BD, RD, toxicity and efficacy studies. In both H69 and H727 lung xenograft tumor models, we showed that following TAT with ²²⁵Ac-DOTATATE, all tumors disappeared and then re-grew, demonstrating significantly increased time to experimental endpoint relative to controls. However, chronic kidney toxicity was observed. Therefore, co-injection with amino acids and fractionated administrations, as has been required for clinical treatment with ¹⁷⁷Lu-DOTATATE, will likely be needed to reduce renal toxicity and generate complete and durable responses.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A process for manufacturing an alpha emitting radiopharmaceutical solution comprising:
combining DOTATATE having a chemical formula 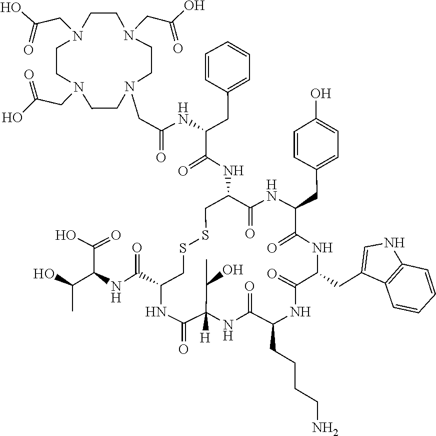 [SMILES: C[C@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O]
with an alpha emitting radionuclide and at least one stabilizer against radiolytic degradation in an aqueous medium to form a solution;
heating the solution to a temperature of greater than about 50 degrees Celsius for a period of at least 30 minutes to form a chelate solution; and
cooling the chelate solution to about 25 degrees Celsius and maintaining at said temperature until secular equilibrium is reach for the alpha emitting radionuclide.

2. The process of claim 1, further comprising optionally checking the radiochemical purity of the chelate solution upon reaching secular equilibrium for the alpha emitting radionuclide.

3. The process of claim 2, wherein the radiochemical purity is determined by instant thin layer chromatography (ITLC) with gamma counting, radio-thin layer chromatography (radio-TLC) or radio-high performance liquid chromatography (radio-HPLC) with gamma counting of fractions.

4. The process of claim 1, wherein the alpha emitting radionuclide is selected from the group consisting of ²¹¹At, ²¹²Bi, ²¹²Pb, ²¹³Bi, ²²³Ra, ²²⁴Ra, ²²⁵Ra, ²²⁵Ac, and ²²⁷Th.

5. The process of claim 1, wherein the alpha emitting radionuclide is ²²⁵Ac.

6. The process of claim 1, wherein the alpha emitting radionuclide is present in a final concentration in the chelate solution that provides a volumetric radioactivity of at least 100 MBq/mL.

7-9. (canceled)

10. The process of claim 1, wherein the chelate solution is maintained at about 25 degrees Celsius for at least 4 hours, at least 8 hours, at least 12 hours, at least 18 hours, or at least 24 hours until secular equilibrium is reached.

11. The process of claim 1, wherein the at least one stabilizer comprises gentisic acid or salts thereof, ascorbic acid or salts thereof, methionine, N-acetyl cysteine, histidine, melatonin, ethanol, Se-methionine, or combinations thereof.

12. The process of claim 1, wherein the at least one stabilizer comprises gentisic acid or salts thereof, ascorbic acid or salts thereof, or combinations thereof.

13. The process of claim 1, wherein the at least one stabilizer is present in a final concentration in the chelate solution of at least 0.2 mg/mL.

14. (canceled)

15. The process of claim 1, further comprising filtering the chelate solution to remove any residual solid components prior to administration to a subject.

16. An alpha emitting radiopharmaceutical composition prepared by the process of claim 1.

17. A method for treating cancer in a subject comprising administering a therapeutically effective amount of a radiopharmaceutical composition of claim 16 to the subject.

18. The method of claim 17, wherein the cancer is a neuroendocrine tumor.

19. The method of claim 18, wherein the neuroendocrine tumor is a functional neuroendocrine tumor or a non-functional neuroendocrine tumor.

20. (canceled)

21. The method of claim 18, wherein the neuroendocrine tumor is selected from functional carcinoid tumor, insulinoma, gastrinoma, vasoactive intestinal peptide (VIP)oma, glucagona, serotoninoma, histaminoma, ACTHoma, pheocromocytoma, somatostatinoma, and non-small cell lung cancer (NSCLC).

22-24. (canceled)

25. The method of claim 18, wherein the neuroendocrine tumor is refractory to treatment.

26. The method of claim 25, wherein the neuroendocrine tumor is chemorefractory or resistant to treatment with a somatostatin analog, a kinase inhibitor, or an inhibitor of the PD-1/PD-L1/CTLA-4 pathway.

27-29. (canceled)

30. The method of claim 18, wherein the radiopharmaceutical composition is administered in combination with one or more additional therapeutic agents.

31-32. (canceled)

33. A kit for preparing a radiopharmaceutical composition according to the process of claim 1, the kit comprising:
DOTATATE;
an alpha emitting radionuclide; and
at least one stabilizer against radiolytic degradation.

34. (canceled)


SEQUENCE LISTING (4 sequences)

SEQ ID NO: 1 - DNA, 20 bp, Artificial Sequence
  Synthetic construct
aaccagacagagccgtacta

SEQ ID NO: 2 - DNA, 18 bp, Artificial Sequence
  Synthetic construct
gcatagcggaggatgaca

SEQ ID NO: 3 - DNA, 22 bp, Artificial Sequence
  Synthetic construct
gctggcttcccttctacatatt

SEQ ID NO: 4 - DNA, 20 bp, Artificial Sequence
  Synthetic construct
gaggaccaccacaaagtcaa